(19) United States
(12) Patent Application Publication
(10) Pub. No.: US 2005/0059046 A1
(43) Pub. Date: Mar. 17, 2005

(54) Methods and systems for the analysis of biological sequence data

(75) Inventors: James LaBrenz, San Francisco, CA (US); Jon M. Sorenson, Palo Alto, CA (US); Curtis Gehman, Burlingame, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 10/871,081

(22) Filed: Jun. 18, 2004

Related U.S. Application Data
(60) Provisional application No. 60/479,332, filed on Jun. 18, 2003.

(51) Int. Cl. C12Q 1/68

(52) U.S. Cl. 435/6; 702/20; 204/450

(57) ABSTRACT

Nucleic acid sequence determination is a method whereby peaks in data traces representing the detection of labeled nucleotides are classified as either noise or specific nucleotides. Embodiments are described herein that formulate this classification as a graph theory problem whereby graph edges encode peak characteristics. The graph can then be traversed to find the shortest path. Various embodiments formulate the graph in such a way as to minimize computational time. In various cases it is desirable that such classification allow for the possibility of mixed bases in the nucleotide sequence. Embodiments are described herein that address the classification of mixed-bases. Embodiments are also described that detail methods and systems for processing the data in order to make the classification step robust and reliable.

### CROSS-REFERENCE TO RELATED APPLICATIONS

This application claims priority from U.S. Provisional Application No. 60/479,332 filed on Jun. 18, 2003, which is incorporated herein by reference.

### FIELD

The present teachings relate to computer-based methods and systems and media for the analysis and evaluation of biological sequence data.

### BACKGROUND

DNA sequencing systems, such as slab-gel and capillary electrophoresis sequencers, often employ a method wherein DNA fragments are separated via migration in a separation medium. Usually labels, e.g., fluorescent dyes, associated with each of the separated fragments are read as the fragments pass through a detection zone. The result is a series of traces, sometimes referred to as an electropherogram, where each trace relates the abundance of the labels over time. Interpretation of the peaks in each trace leads to a determination as to the genetic sequence of the sample. Such interpretation, sometimes referred to as base calling, can be carried out manually or in an automated fashion (e.g., using a programmed computer). The method of interpreting the signal is central to the base calling process and can greatly affect the quality of the results.

### SUMMARY

According to various embodiments, nucleic acid sequence determination is formulated as a problem in graph theory. Theory and implementation of a solution are discussed and described herein.

Various embodiments of the present teaching provide a method that applies graph theory to determine the nucleotide sequence of a sample assayed on an electrophoresis machine, including: obtaining traces from a plurality of channels of an electrophoresis detection apparatus, preprocessing the traces, applying a graph theory formulation to classify one or more of the peaks and reporting the peak classifications.

Other embodiments include methods wherein the graph theory formulation encodes information about peak characteristics and possible paths through the graph in the edge weights.

Various aspects relate to methods of forming the graph in a manner that reduces computational complexity, including: using a window to select a portion of the peaks, designating each peak as a node, connecting the nodes with edges wherein the edges encode a transition cost between the connected nodes, and determining a shortest path through the graph to classify peaks.

Further aspects relate to a method of determining the presence of mixed bases, including: creating additional nodes in the graph when two nodes are within a specified distance so as to appear coincident, and designating the additional nodes as mixed bases that encompass some combination of the coincident peaks.

Additional aspects relate to program storage devices readable by a machine, embodying a program of instruction executable by the machine to perform method steps for nucleic acid sequence determination. In various embodiments, the method steps include: obtaining data traces from a plurality of channels of an electrophoresis detection apparatus wherein the traces represent the detection of labeled nucleotides, preprocessing the data traces, identifying a plurality of peaks in the data traces, applying a graph theory formulation to classify one or more of the peaks, and reporting the peak classifications as a nucleotide sequence.

### DESCRIPTION OF THE DRAWINGS

The skilled artisan will understand that the drawings, described below, are for illustration purposes only. The drawings are not intended to limit the scope of the present teachings in any way.

FIG. 1 illustrates electrophoretic traces that can result from the separation of fragments.

FIG. 2. FIG. 2a-f illustrates methods contemplated by embodiments of the present teachings.

FIG. 3 illustrates an example of a filtered power spectrum used to estimate spacing.

FIG. 4 illustrates an example of a spectral function used to estimate peak width. The function is represented by the solid line. A function is fit to the portion of the curve that represents the signal.

FIG. 5 illustrates a method for peak detection contemplated by one embodiment of the present teachings.

FIG. 6 illustrates a method for peak cluster identification contemplated by one embodiment of the present teachings.

FIG. 7 illustrates exemplary data showing two hypotheses for the composition of a peak cluster.

FIG. 8. FIG. 8a illustrates an example of electrophoretic data with four different labels—one attached to each of the nucleotides in the sample. The solid trace corresponds to the nucleotide adenine (A), the dashed trace corresponds to the nucleotide thymine (T), the dotted line corresponds to the nucleotide guanine (G) and the dash-dot line corresponds to the nucleotide cytosine (C). Vertical lines indicate the positions of expected peaks. FIG. 8b illustrates a directed acyclic graph representing the peaks identified from the data in FIG. 8a. A window is used so that only the final six peaks in the sequence are under consideration. The lettered nodes represent the corresponding peak in the electropherogram of FIG. 8a. The numbers associated with the edges between the nodes encode transition costs between the nodes.

FIG. 9. FIG. 9a illustrates an example of an electrophoretic data with four different labels—one attached to each of the nucleotides in the sample. The solid trace corresponds to the nucleotide adenine (A), the dashed trace corresponds to the nucleotide thymine (T), the dotted line corresponds to the nucleotide guanine (G) and the dash-dot line corresponds to the nucleotide cytosine (C). Vertical lines indicate the positions of expected peaks. A mixed base is present at the fourth peak position. FIG. 9b illustrates a directed acyclic graph representing the peaks identified from the data in FIG. 9a. A window is used so that only the final five peak positions are under consideration. The lettered nodes represent the corresponding peak in the electropherogram of FIG. 9a. The numbers associated with the edges between the nodes encode transition costs between the nodes. An additional node (M) is included indicating the hypothesis that the base at the fourth peak position is a mixed base and is composed of the nucleotides adenine and cytosine.

FIG. 10 illustrates sample results for signal exhibiting poor resolution.

FIG. 11 illustrates sample results for mixed-base data.

FIG. 12 is a block diagram that illustrates a computer system, according to various embodiments, upon which embodiments of the present teachings may be implemented.

FIG. 13 illustrates exemplary data showing the output of a matched-filter correlation.

FIG. 14 illustrates electrophoretic data showing the presence of mixed-base peaks.

FIG. 15 illustrates a class diagram showing an embodiment of the present teachings for data representation.

### DESCRIPTION

Definitions

Channel is defined as one of the traces in an electropherogram signal. A channel typically refers to one label used in identifying a nucleotide.

Sample is defined as the DNA sample under investigation.

Sample space is a term used to indicate that a characteristic belongs to a sample. For example, a peak is said to reside in sample space if its presence is directly attributable to the sample. Thus if a peak indicates the presence of an adenine nucleotide at some position and there is in fact an adenine nucleotide at that position, then the peak is said to belong to the sample space.

Noise space is a term used to indicate that a characteristic does not belong the sample space. For example, if a peak indicates the presence of an adenine nucleotide at some position and there is in fact no adenine nucleotide present at that position, the peak is said to belong to the noise space. There are a variety of causes for noise space peaks. Some common causes are sample preparation error, unincorporated dye, instrument error, limits on instrument resolution and operator error.

A. Description of Base Calling System

The teachings herein relate at least in part to a base calling system for the determination of a DNA sequence. Different types of instrumentation can be used for collecting raw sequencing data. Instruments are available from Applied Biosystems, Amersham, Li-Cor and other companies. These systems are often referred to as sequencers. Many of these systems utilize labels that are attached to DNA fragments.

These DNA fragments are formed from a sample and separated according to mobility. In various systems, slab gels and polymer filled capillaries are used for the separation and an electric field is used to effect migration of the fragments in these media. Reading of the labels over time produces a signal that is comprised of a trace for each channel where a channel corresponds to a respective label (e.g., a dye). In some systems additional channels are included that can yield information in additional to the channels corresponding to the nucleotides. This information can be used for better estimating spacing or other parameters that may render sample analysis easier. Such a system is contemplated in U.S. patent application Ser. No. 10/193,776 (publication no. 03-0032042), assigned to the assignee hereof, which is incorporated by reference herein in its entirety.

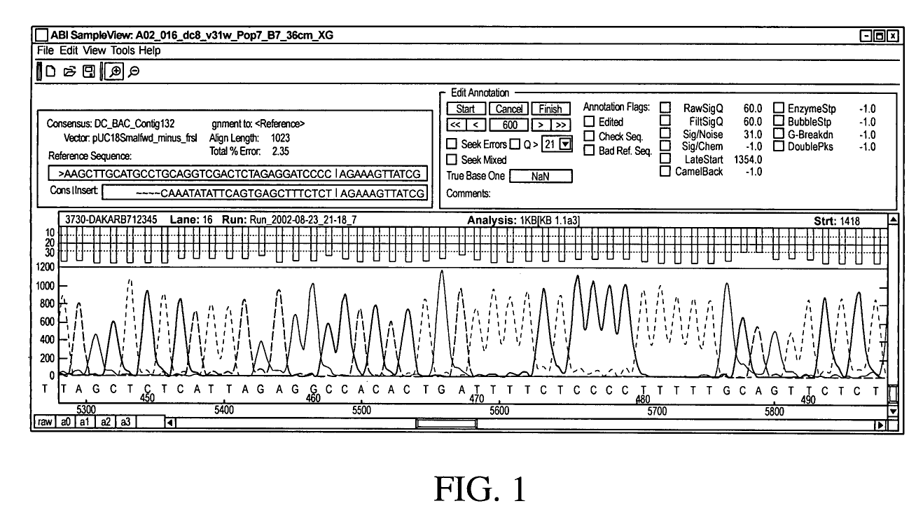

FIG. 1 shows data from a typical sequencer. Here four traces are present. Each trace represents a channel. Each channel represents a different label and each label corresponds to a different nucleotide. This data is taken from the middle of a sample run and would be considered by one skilled in the art to be of good quality. Good quality can be assessed by the regularity of the spacing and the distinctness of the peaks. The base calls appear under each peak as the letters A, C, G, and T. Quality values appear above the peaks with a longer bar representing a higher quality value. The lower set of numbers on the x-axis represent the scan number and the higher set represent the base number. The x-axis can also be considered to represent time.

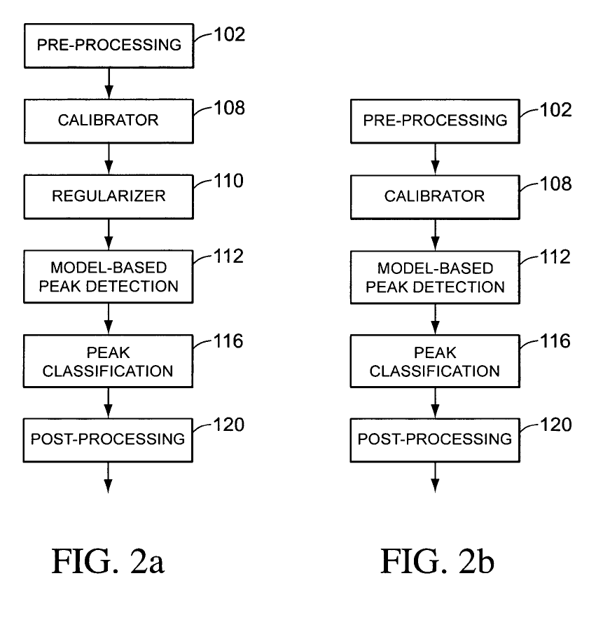

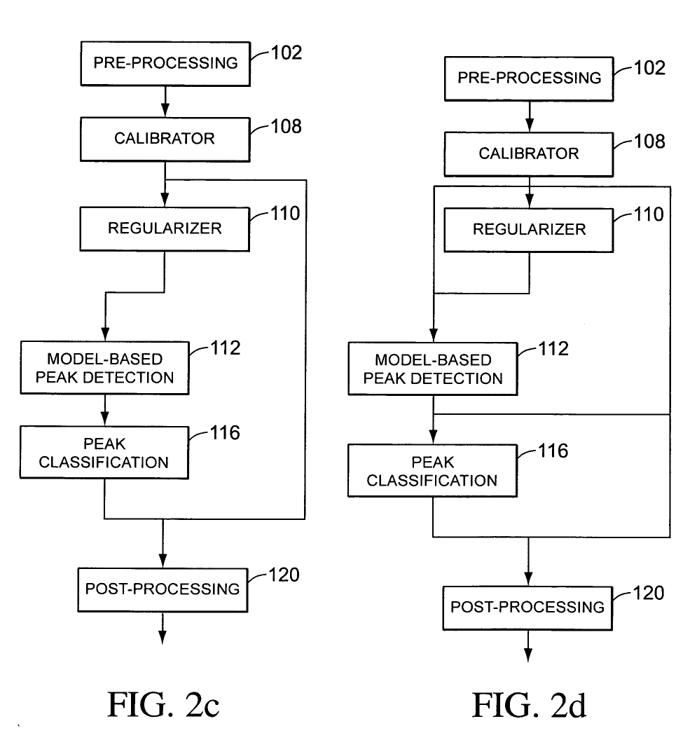

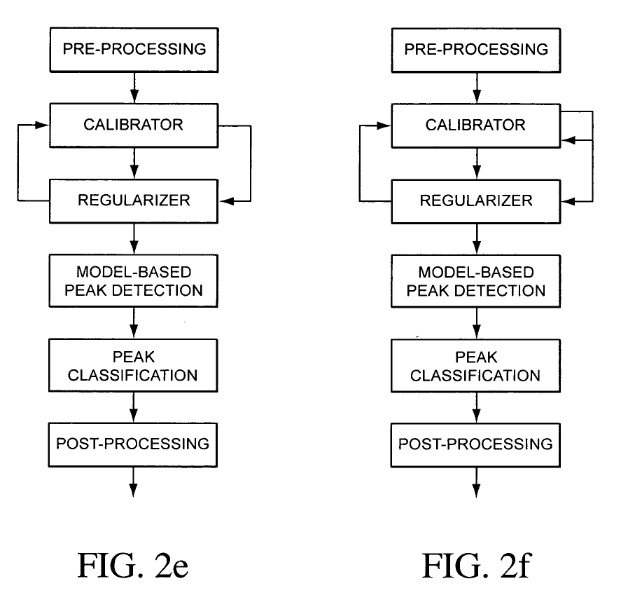

Various embodiments of a base calling system, in accordance with the present teachings, are depicted in FIGS. 2a-f. Herein, the system is described as comprising several functional modules. Modules performing similar function in FIGS. 2a-f are numbered identically. Each module broadly categorizes the types of operation(s) that occur in it. For example, the preprocessing module (102) can be comprised of one or more functions that are typically performed on raw sequencing/electropherogram data.

According to various embodiments, a calibrator module (108) can serve to identify parameters for the signal that are useful for subsequent stages as well as for adjusting calibration of certain parameters. These parameters can include, but are not limited to, peak spacing and/or peak width and models of the amplitudes. Estimation of such parameters prior to peak detection can augment peak detection. As well, these parameters can also be used by one or more other modules in the system.

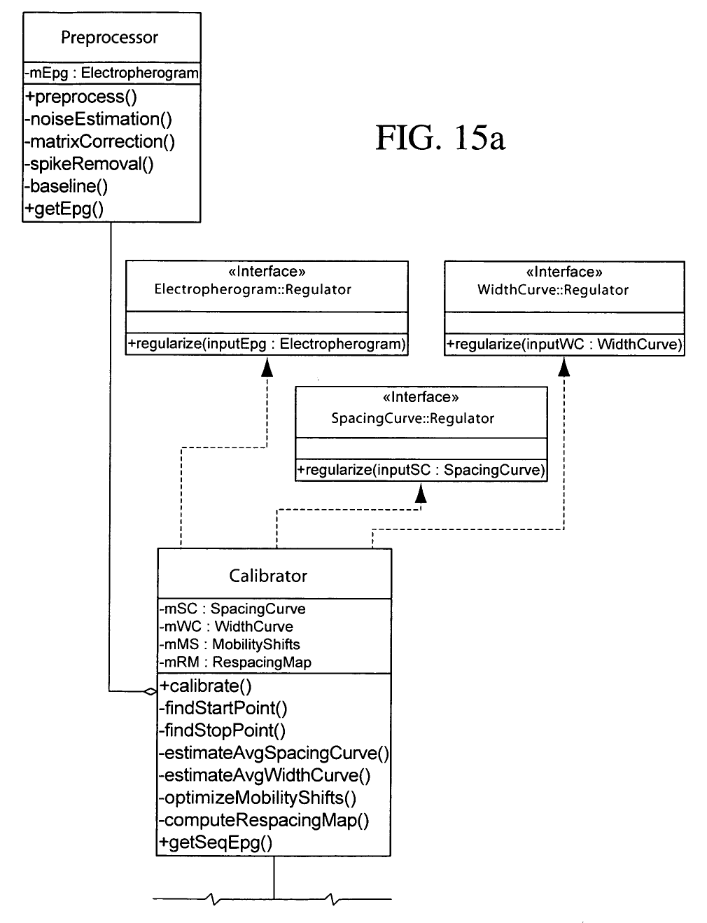

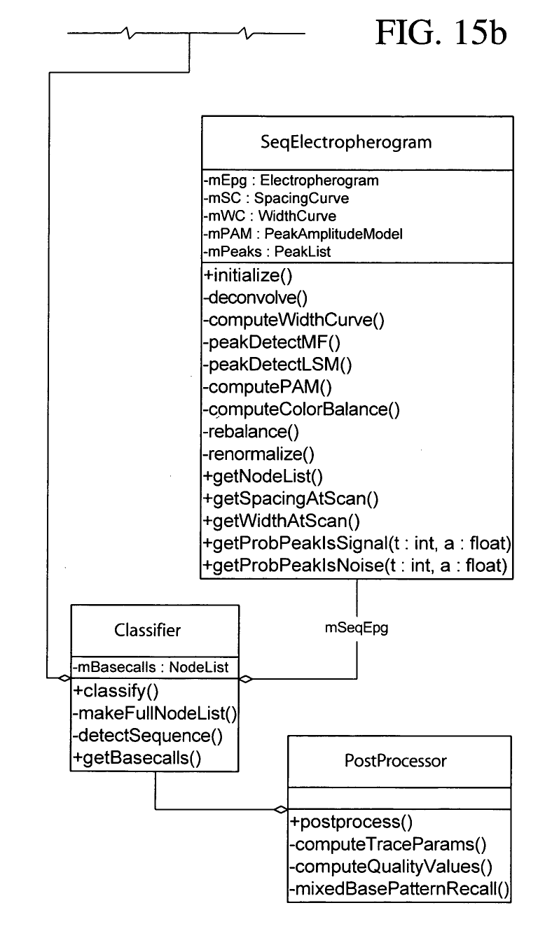

Various embodiments use the output of the calibrator module to provide information for regularizing the data. Raw data and pre-processed data can exhibit large changes in amplitude and other parameters throughout the signal. Estimation of how such parameters change can be used to normalize these changes and produce a signal that is not only more easily analyzed by subsequent processing stages but also more readily interpreted by a human observer. Such a function is performed by the regularizer module (110). When implemented in a computer system, the data, associated parameters and methods can be stored in an class. This can include the raw data, any calibration curves, estimates of the parameters, the base calls themselves, and the functions used to analyze the data. An embodiment of such a class is illustrated in FIG. 15. Any information about the signal and the numerous methods applied to it, as well as their results, can also stored in the class. Regularization via application of the characterizing parameters will often require the update of the contents of this class and can result in multiple representations of the signal (raw, baselined, spectrally corrected, etc.).

In some embodiments, a model-based peak detection module (112) can use information from the calibration module in detecting peaks. In doing so, the peak detection module can identify clusters of peaks, where clusters can have one or more peaks. The peaks can be distinct or, in the case of poor resolution, the peaks can be smeared together. By using estimates of the signal's parameters, a peak cluster can be resolved into its constituent peaks.

In various embodiments, a peak classification module (116) can classify the peaks detected as belonging to sample or noise space. Some embodiments of the system utilize graph theoretic approaches to perform the classification. In forming the graph, for example, peak characteristics, local sequence characteristics, and/or global signal characteristics can be used to define transition weights between the peaks.

Various embodiments use windows of various sizes to traverse the graph.

According to various embodiments, a post-processing module (120) can perform operations typical of base calling systems. These can include, for example, quality value determination. Various embodiments of the systems can use information determined in any of the previous stages in calculating quality values and recalling bases. Examples of such data include, metrics from the optimization of model fitting during peak detection, metrics from any of the signal characterization steps, metrics and parameters generated during the preprocessing step, estimates of noise strength, closeness of paths determined during the classification stage, etc.

FIG. 2a illustrates an embodiment of the system. One skilled in the art will appreciate how information can flow between modules. FIG. 2b illustrates the system without the regularizer module. This does not depart from the teachings as the parameters output from the calibrator are still used in peak detection and subsequent stages. One skilled in the art will appreciate that the information obtained in the calibrator can be used in several different modules. The characterizations of the signal can be used in post-processing as illustrated in FIG. 2c. FIG. 2d shows the Calibrator output being used in peak detection, peak classification and post-processing. One skilled in the art will be able to appreciate that estimated signal parameters can be used in multiple stages of the system which can benefit overall system accuracy.

In FIG. 2c, information from the calibrator module can be used in the post-processing module. In FIG. 2d information from the calibrator module can be used in the post-processing module and in the classification module. As well, information from the model-based detection module can be used in the post-processing module.

One skilled in the art will appreciate that parameter estimation is often an iterative process that can require reestimation of parameters based on updated information. Such a situation is illustrated in FIG. 2e where characterization via the calibrator and regularizer can be looped in order to tighten tolerances on parameter estimates. A similar situation is shown in FIG. 2f where a loop can occur within the calibrator module itself as an estimate of one parameter can trigger reestimation of another parameter which can trigger the need for reestimation of the former parameter.

These figures are intended to illustrate a plurality of information flows in the system and one skilled in the art will be able to envision other configurations that adhere to these teachings.

B. Preprocessing

Signal preprocessing can involve a plurality of stages, many of which are particular to the instrument being used. These functions can include, for example, multi-componenting, baselining, smoothing, size-calling, mobility correction, signal enhancement, signal strength estimation, noise level estimation and filtering. One skilled in the art will have an appreciation of the variety of ways that these operations can be undertaken and the types of typical preprocessing steps that are applied to data. While these functions are grouped in the preprocessing module, the distinction is not intended to limit them to this module only as they can instead be realized at other points in the system. One step that is generally common to many instrument systems in spectral calibration. Embodiments of the present base calling system employ methods for correcting error introduced by calibration methods that may not fully deconvolve the data.

i. Spectral Calibration Error Correction

DNA analysis instruments typically employ multiple fluorescent dye labels as a means of detection. Such methods can require a deconvolution in the spectral dimension of the raw data electropherogram to facilitate further downstream analysis. This process transforms the data from a representation where each channel corresponds to the light emission intensity in a particular spectral filter (i.e., a physical range of light wavelength) to one in which the channels correspond to relative dye species emission intensity.

One skilled in the art will appreciate that the standard mathematical model for such systems is x=As, where the raw data electropherogram x is related to the underlying dye emission electropherogram s through the matrix of dye spectral profiles A. The underlying source signals s, can be obtained via the inverse equation s=A⁻¹x.

Solving the inverse problem M=A⁻enables the spectral deconvolution of the raw data electropherogram. Commonly, the optical detection system of the electrophoresis instrument is first calibrated to obtain the spectral calibration matrix A. Its inverse, M, is computed, and one of the signal processing steps in automated data analysis of electrophoresis experiments can be the so-called multicomponent transformation which is also known as spectral calibration or spectral deconvolution. A common approach to spectral calibration is to run a known set of analytes through the system in such a way that spectral regions represented by a limited number of dye species can be identified. From these regions, the spectra that characterize individual dyes can be computed to construct the matrix A. A method of performing this transformation is contemplated in U.S. Pat. No. 6,333,501 assigned to the assignee hereof, which is incorporated by reference herein in its entirety.

When a calibration is accurate, there may be no need to further correct or modify the multicomponent transformation. However, in some instances, systematic error in the estimates of the calibration parameters, Aᵢⱼ, can lead to corresponding errors in the estimation of the underlying dye signals s. An isolated peak in a given channel of s can result in characteristic smaller positive peaks or negative peaks in other channels. One skilled in the art typically refers to these as pull-up or pull-down peaks. These secondary peak artifacts can be correlated in time with the true peak. This circumstance is often referred to as spectral miscalibration.

A variety of pathologies can lead to spectral miscalibration. Examples of these include noise or unwanted artifacts in the calibration chemistry and differences between the calibration chemistry and analysis chemistries. These can result in the spectral signatures seen by the instrument optics differing slightly from, and temporal drift in the true spectra that is not reflected in, the original calibration. Pull-up peaks can represent additional challenges to downstream analysis algorithms as positive pull-up peaks can look identical in shape to the peaks arising from the DNA fragment molecules in other channels. Pull-up peaks can increase the error rates in base calling or allele calling as well as produce errors in baselining algorithms. The problem of miscalibration can be particularly vexatious in the case of mixed-base calling.

Some embodiments counter the effect of miscalibration by introducing a transformation via a matrix B to correct for effects not considered by the original spectral calibration matrix A. Similar to the spectral calibration process, pull-up correction of a deconvolved electropherogram \overline{s} that contains pull-up error can be modeled as arising from a linear transformation applied to the underlying electropherogram s, by the relation ŝ=Bs. The pull-up matrix B can be defined to have ones on the diagonal and carrying dimensions n_{D}×n_{D}, where n_{D} represents the number of dyes in the system. For low levels of pull-up the off-diagonal elements are often small. Given B, a pull-up corrected signal can be obtained by application of the formula s=B⁻¹\overline{s}.

Various embodiments provide a method for determining the matrix B from an input electropherogram \overline{s} that contains a moderate amount of pull-up error, typically approximately ≦10%. The method can be considered to be a case of blind deconvolution.

Various embodiments form the pull-up matrix B by finding regions of the data that represent pure dye peaks with small pull-up peaks underneath, positive or negative, estimating the pull-up ratio r for that combination of channels and constructing a correction matrix. This process can be performed by one or more of the following steps,
1. Initialize the correction matrix, B=I
2. Estimate the pull-up of a minor dye j under the major dye i
3. If the degree of pull-up surpasses some threshold, estimate the pull-up fraction r and set Bᵢⱼ=r.
4. Apply the transformation, s=B⁻¹ŝ, where ŝ is the miscalibrated signal.

Various embodiments estimate the pull-up by first detecting candidate points tₖ for pull-up estimation. One skilled in the art will appreciate that there are several methods for candidate detection. Various embodiments perform candidate detection via analysis of rank on a sub-matrix of the electropherogram around each scan t. Such a method is contemplated in U.S. Pat. No. 6,333,501. Various embodiments use methods based on the correlation between major and minor channels. In such a method, the elements of an electropherogram matrix sⱼₜ can be denoted sⱼ(t), where j is a channel index, and t is a time or time-related index, t=0, . . . , n_{T}−1. Regions of pull-up can be identified by computing the difference of the second derivative in a minor channel j to produce a signal dⱼ where dⱼ(0)=0, and dⱼ(t)=s″ⱼ(t)−s″ⱼ(t−1). Generally, a set of scans, {tₖ}, that meet the following criteria, |s″ᵢ(tₖ)|>|s″ⱼ(tₖ)|>α₁ and |dⱼ(tₖ)|/|s″ⱼ(tₖ)|<α₂ where α₁ and α₂ are arbitrary constants can be found. Suitable values for these parameters are α₂=0.01; α₁=max(1, σ), where σ is ½ times the maximum of the estimated baseline noise across the color channels. Clustering can be used to locate several points with similar pull-up angle characteristics. If the cardinality of the set {tₖ} exceeds some threshold, NT, the calculation of r can proceed. For each tₖ, the angles and magnitude are computed as follows. Setting a=s″ⱼ(tₖ) and b=s″ᵢ(tₖ). θₖ can be defined as the arcsin of the ratio of a\hat{ }2 to b\hat{ }2 and ηₖ can be defined as the 2-norm of a and b. The pull-up ratio r can be computed using a weighted mean of the angle. Some embodiments first find a median value \hat{θ} of the angles θₖ and create a subset of the angles θₖ by finding the θₖ that fall within some Δθ of the weighted mean of the angles. This subset is called θ₁. The pull-up ratio can then be computed by the following equation,

$$r = {\sum }_{l} {θ}_{l} {η}_{l} / {\sum }_{l} {η}_{l} .$$

Some embodiments define a quality q≡|r|/δθ where δθ is a weighted standard deviation of the retained values. If the standard deviation δθ>δθ_{T} or q<q_{T} the estimate can be rejected and the assignment r=0 can be made. Various embodiments use miscalibration correction for estimation of spectral miscalibration, which can provide a useful instrument diagnostic. Suitable value for these parameters are δθ_{T}=0.075 and q_{T}=1.5. Once a value for r is determined, the substitution Bᵢⱼ=r can be made.

C. Calibrator

The calibrator performs estimates of parameters of the base calling system. These estimations are performed early in the system and find utility in several different stages. They can be used to augment the peak detection process, in peak identification as well as in post-processing. They also facilitate regularization of the data. Their early estimation facilitates the use of model-based methods throughout the system. The calibrator can also update calibration curves in several different ways that increase the reliability of the curves.

i. Calibration curve adjustment

Existing base callers often use calibration data stored in a file that describes the spacing and width of peaks as a function of time or position in sequencing electropherograms for a particular sequencing platform. Such calibrations often take into account parameters such as the instrument type, separation medium, assay specifics or other parameters. These calibration data are often referred to as calibration curves. Variations in physical conditions or sample preparation processes can lead to variations in the actual spacing or widths of peaks in the collected data. The accuracy of applying these curves can be increased by tuning them frequently and even on a run-to-run basis. Many of the parameters such as spacing and widths of peaks in electropherograms can be estimated by the base caller software. Such estimates can sometimes be inaccurate. Calibration curves are often employed where dynamic estimates fail. Successful estimates, can, however, be used to scale or otherwise adjust the stored calibration curves in order to improve their accuracy. These adjustments to the calibration curves can be useful even in regions where the dynamic estimates fail.

Some embodiments assume that the basic shape of a calibration curve remains the same but that its scale may vary from run-to-run and that a global, uniform correction can be applied to the curve. Determination of a parameter over a section of the data can be used to scale the calibration curve via multiplication of the curve by the ratio of the parameter estimated at a point to the value of the calibration at the same point.

Various embodiments of the present teachings nonuniformly scale the calibration curves to provide a better fit of the estimates by assuming the existence of an arbitrary original calibration curve c₀(t) and a set of corresponding estimates S={(tᵢ, yᵢ):i=1, 2, . . . , n} which are derived from the current sample. Typically, c₀(t) is represented as a vector of discrete samples. To evaluate c₀(t) for an arbitrary t, one of a number of standard interpolation methods can be used. In general a new calibration curve c₁(t) is sought that fits the estimates in S. Some embodiments let
c₁(t)=f(t)c₀(t) (15)

For c₁(t) to fit S, f(t) can fit a derived set of estimates R={(tᵢ, zᵢ)}, where

$${z}_{i} = \frac{{y}_{i}}{{c}_{0} ( {t}_{i} )} .$$

Some embodiments fit R with a simple function, such as a low-order polynomial. Using a polynomial form can enable determination of f(t) rapidly by a linear least squares fitting method. Once f(t) is determined, Equation 14 can be used to compute the curve c₁(t) that fits the estimates S.

ii. Start Point Detection

Accurate detection of features representing the shortest fragments in an electropherogram signal can increase the amount of usable data in a sequencing run. This position is often referred to as the start point. The start point can provide a reference point for calibration. For example, spacing estimates and mobility shifts can depend on the start point and poor estimation of the start point can lead to errors in these functions.

One skilled in the art will appreciate that accurate detection of the start point in clean data can be straightforward. However, it will also be appreciated that a diversity of pathologies commonly found in DNA sequencing data can make start point detection difficult and prone to error.

Various embodiments of the present teachings apply morphological filters to data in order to determine the start point. An opening, \hat{M}h, can be envisioned as cutting isolated positive features, such as peaks, narrower than a given scale h, down to the signal level of their neighborhood. Conversely, a closing filter Mₕ, can be considered to raise isolated negative features, such as troughs, narrower than a given scale h, to the level of their neighborhood. The scales of these morphological filters can be set according to characteristics of the DNA fragment features in the electropherogram such as peak spacing, \overline{s}, and average peak width, \overline{w}. Such a process can be described by one or more of the following steps,
1. Construct profile functions of the electropherogram
2. Calculate thresholds
3. Find where the profiles first rise above the thresholds
4. Start point validation

Some embodiments of the present teachings sum an electropherogram signal, xᵢ(t), where i is an integer ranging from 1 to n_{channel}, across channels, to yield a new signal, p₀(t). On this new signal an opening filter can be used to remove sharp spikes as follows.
pᵢ(t)=\hat{M}ₕ_{₁}P₀(t) (1)
One suitable configuration of the opening filter sets the scale for the opening filter smaller than the narrowest DNA fragment feature. One skilled in the art will appreciate that if the scale is made too small, one risks retention of fat spikes. Typically, automated DNA sequencing instruments are engineered to generate electropherograms in which the DNA fragment features are at least 7 scans wide. In such cases, some embodiments set a suitable value for the scale is h₁=5 scans.

Various embodiments contemplated herein construct a profile that runs near the tops of most peaks by applying a closing filter as follows, p₂(t)=Mₕ_{₂}p₁(t). This can remove the drops towards the baseline that can be found between well-resolved peaks. A suitable range for the scale parameter h₂, is a few times the expected peak spacing. One skilled in the art will appreciate that electropherogram data often exhibits features in advance of the start point that do not correspond to DNA fragments and that if h₂ is too large, the drop in signal between such “false” features and the start point can be eliminated. This can lead to a premature start point. Some embodiments set a suitable value for h₂, as five times the expected peak spacing, h₂=5\overline{s}.

When broad isolated positive features exist, some embodiments apply an opening filter can be applied as follows, p₃(t)=\hat{M}ₕ_{₃}p₂(t). If drops to the baseline have been previously removed, the scale parameter h₃ can be much larger than the typical peak width \overline{w}. A suitable value for the upper limit of this range can be the length of the region containing strong DNA features. For some classes of data, this length can be hundreds of peak spacing. For short PCR products, the length can be arbitrarily short, though other considerations can lead to a lower limit of 50 to 100 bases. If one value of h₃ is to apply to all classes of data, some embodiments make it shorter than the shortest length of DNA expected. Even in cases where the DNA is longer, it is possible that some broad drops were not removed previously. Given these considerations, a suitable value can be h₃=20\overline{s}. The profile can be baseline shifted by subtracting the minimum from all elements to yield a final profile p(t)=p₃(t)−min p₃.

While the aforementioned filtering operations are listed sequentially, one skilled in the art will appreciate that it in certain circumstances it may be desirable to change the order or employ one or more of each type of filter. It will be appreciated that such reordering, omission of a morphological filter or multiple applications of a filter is within the spirit of these teachings.

Various embodiments of the present teachings construct a slope profile by differencing between samples that are δt apart as follows, q(t)=p(t+δt)−p(t). A suitable value is δt=\overline{s}/2 where \overline{s} is an estimate of peak spacing.

In various embodiments of the start point detection method, the start point can be defined as the point where the profile crosses a threshold, \overline{p}. The threshold should exceed the background noise prior to the real start point and be below the profile after the real start point. One skilled in the art will appreciate that there are various ways of computing the noise threshold. One suitable technique involves the use of a difference square statistic. Given an estimate of the noise level σ, a lower limit for the threshold can be determined as a multiple of the noise level u=αₙₒᵢₛₑσ. The value of αₙₒᵢₛₑ may not be readily determined a priori as it can depend on the method used to estimate the noise. Typical values are in the range, 3≦αₙₒᵢₛₑ≦10. Some embodiments make the threshold lower than the maximum of the profile p(t). An upper limit can be defined as ν=α_{signal} max p. The value of a_{signal} may not be able to be determined a priori as it may depend on the method used to estimate the noise and the desired sensitivity. For example, this may be of concern in data where the amplitude of the early DNA features rises slowly out of the noise. One suitable value is α_{signal} =0.060.

One skilled in the art will appreciate that in clean data, generally, u<ν, and that a suitable choice for the threshold \overline{p} should lie between these two limits. Various embodiments use the mean of these two values. As well, for data in which the DNA features are weak, the inequality may not hold. In such cases, the noise-based limit can be used as the threshold value. Various embodiments calculate a slope threshold \overline{q}, by dividing \overline{p} by δt.

The start point can be determined by locating the point where the profile crosses \overline{p}, where the slope profile crosses \overline{q} or by a combination of these conditions. This latter case is represented by the equation t₀=min {t|p(t)>\overline{p} and q(t)>\overline{q}}. Various embodiments employ start-point validation techniques. One such technique involves forward scanning until the profile exceeds one half of its maximum and then scanning backwards to a point, t₁, where the profile falls back below β\overline{p}, where is β≧1 but not much greater than 1. The start point can be reported as the maximum of t₀ and t₁. This step can be used to mitigate the possibility that the threshold crossing is due to a statistical fluctuation of the noise or a broad but weak isolated feature that was not removed by a morphological operation.

iii. Model Change Detection

A given model or parameterization of a model may not be suitable to cover the entire range of the data. It can be useful to detect points where model parameters change. A Model Change Point (MCP) can be defined as an approximate point where a model, or equivalently, the parameters of a model that describe them need to be modified in a non-trivial way in order for the model to fit the data. In other words, a simple functional form ν(t) may not produce a good “fit” to the data across the MCP. Some common examples are the following. Start Point: The location of the first peak in the DNA sequence. Prior to the start point, nuisance parameter models are generally undefined. Stop Point: In sequencing short PCR fragments, the sequence of valid DNA peaks generally stops prior to the end of the electropherogram. Enzyme Drop: A sudden drop in peak amplitude, due to local sequence context that is problematic for the enzymatic extension reaction. Compression: A localized compression (and possibly subsequent expansion) of DNA peak spacing, caused by local sequence similarity. In-del Polymorphism: In re-sequencing of genomic DNA, an insertion/deletion polymorphism between the two template molecules will produce a second sequence downstream of the mutation point. Determination of an MCP can be useful to determining any of the above examples. MCP determination can also be useful for detecting points where models change during the determination of calibration curves. In determining distribution coefficients for nuisance parameters, one strategy is to adopt simpler heuristic forms for the ν(t) and to fit the data by sections. Piecing together linear or polynomial fits is an example of this approach.

Some, model parameters are referred to as nuisance parameters. This moniker is used to reflect the fact that the parameters are different from the parameter that is of primary importance in base calling—that is the base call itself. MCPs can be determined by one or more of the following steps.
Identification of a method to estimate a parameter of interest.
Identification of statistical model of the parameters.
Detect MCPs based on changes in model or model parameters.

Typical nuisance parameters of interest include peak amplitude, peak spacing and peak width, which can be, represented over a region as means designated as \overline{a}ⱼ(t), \overline{s}(t) and \overline{σ}ⱼ(t) respectively. If each variable is modeled as independent and normally distributed, each mean parameter can be associated with a standard deviation. These are aⱼ(t), [symbol] ⱼ(t), νⱼ(t) respectively. One skilled in the art will appreciate that more complex models can also be constructed, including ones that depend on local sequence context.

The example of amplitude change detection is given to illustrate application of the method. The method can be used to determine the electropherogram's start point and to identify regions where the amplitude changes substantially. It can also be used to determine the point where models or their parameters change substantially. Such a step is useful for providing estimates during model fitting.

Prior to the start point, the model can be \overline{a}=0; subsequent to the start point, it is some function \overline{a}(t). The example proceeds by estimating a sequence of approximate peak amplitudes {aₖ} from the electropherogram. This can be considered to be a new time series, in time index k that is drawn from a model amplitude distributions.

Various embodiments employ a method as follows. A baselined electropherogram ŷ(t) is computed by estimating the background source bⱼ(t) and subtracting it from yⱼ(t), so that ŷⱼ(t)≈xⱼ(t)+nⱼ(t)+wⱼ(t). A signal profile, z(t), is computed. Exemplary methods include but are not limited to the following techniques;

$$z ( t ) = {\sum }_{j} {\hat{y}}_{j} ( t ) or z ( t ) = {max}_{j} {\hat{y}}_{j} ( t ) .$$

A closed profile, \check{z}( ), is computed by applying the morphological closing filter Mₕ. An appropriate value for the scale parameter h should be large enough to eliminate the drops (troughs) between peaks, and small enough to maintain some of the variability that would be described by the model. An appropriate value is, h˜κ₁\overline{s}, where κ₁ is an arbitrary value that can be close to 1. Some embodiments, may use the same closed profile computation described for start point detection, to eliminate outlier data, such as spikes or chemistry blobs. The amplitude time series {aₖ} is formed by sampling \check{z} ( ) at an interval tₖ₊₁−tₖ=κ₂\overline{s} where κ₂ is an arbitrary value that can be close to 1.

Some embodiments employ alternate methods of estimating {aₖ} such as sampling a list of peaks detected in the electropherogram however it can be advantageous to decrease the reliance on other computations by using the above method and eliminating the need for peak detection.

MCPs can be detected in the series {aₖ} by employing a hypothesis testing method. An appropriate choice is the Generalized Likelihood Ratio Test (GLRT). For example, the amplitude model can be defined as N(\overline{a}(t), α(t))=N(\overline{a}₀, α₀), t<t₀, N(\overline{a}, α₁), t≧t₀. If (a₀, α₀)˜Σ, the scale of the electrical noise at the baseline, it can be postulated that this reflects an absence of DNA signal prior to the change point, and subsequent to the change point the peaks have a constant mean amplitude a₁ and constant variance α₁². As a simplification, a₁(t) can be assumed to varying slowly and can thus be approximated over appropriate regions as a constant value. This can be denoted as a length scale, κ₃\overline{s}, in the original electropherogram, and by κ≡κ₃/κ₂ in the time series {aₖ}. The detection method can be generalized to accommodate other functional forms over intervals of length κ. The GLRT as employed for the change detection on the series aₖ may employ two averages and a test statistic.

Some embodiments compute a localized GLRT change detection by first computing the averages which reflect a local metric of the parameter of interest, in this case amplitude, on either side of a proposed model change point. This metric can be the average of the amplitudes of the values in the windows k₁ to k₀ and k₀+1 to k₂ where k₀ is the proposed model change point. The test statistic can be computed as Δ[k₀]=(Â₁[k₀]−Â₂[k₀])²/(\hat{α}²/κ) where \hat{α}² is a scale parameter representative of the amplitude variance.

Some embodiments define a change point can be defined as a point where

$${max}_{{k}_{0}} Δ [ {k}_{0} ] > γ .$$

If the model variances a₀² and a₁² are known a priori, the scale parameter \hat{α}² and the threshold γ can be used to set a probability of false alarm from the detector as prescribed in Kay, SM, Fundamentals of statistical signal processing: Detection theory vol. II, Prentice Hall 1998. The scale â² can be estimated from the time series {aₖ}. A method of performing this estimation involves computing the difference series dₖ=aₖ₊₁−aₖ and determining the set of indices {l|d₁˜0}. The non-zero changes {dₗ} can be sorted and the scale can be computed from a representative section such as the middle section of the signal, which can be approximately the middle 67%. If the number of non-zero differences is less than some minimum number, a large number can be returned, thus effectively disabling the change detection. This method can be tuned to greater or lesser sensitivity by adjusting the threshold parameter γ, and various choices can be made as to what constitutes the best tuning. All local maxima in the test statistic Δ[k₀] that exceed the threshold can be reported amplitude change points. This method can be used to detect multiple change points, including the start point, stop point (e.g., for short PCR fragments), and enzyme-drop events. The method can be applied to parameters of interest other than amplitude. The MCP detection method enables up-front segmentation of the electropherogram, into regions that can be fitted by smooth functional forms.

iv. Estimation of Peak Spacing

Estimation of the spacing of peaks in an electropherogram signal can allow the system to establish the location of expected peaks. Peaks at or near expected locations generally are strong contenders for membership in sample space. Estimating peak spacing, according to the present teachings, can be carried out prior to detecting peaks in the signal. Freedom from a priori peak detection can provide an advantage, for example, when the signal is littered with peaks that belong to the noise space. Estimates of peak spacing can be used in peak identification and classification.

In various embodiments, peak spacing can be estimated using Fourier techniques. Estimation proceeds, in some embodiments, by selecting an interval of the data throughout which the average peak spacing is approximately constant. This can be accomplished, for example, by selecting data that is in the middle of the run, although the precise location is not crucial. For the data in this interval, the following steps can be executed:
correct for mobility shifts and sum across channels
compute high-pass filtered power spectrum
find global maximum in filtered power spectrum
compute corresponding spacing and estimate reliability

Mobility shift correction can assist in producing a single (real-valued) time signal that has generally uniformly spaced peaks that reflect the average spacing of bases in the electropherogram. This can require that some estimates of the mobility shifts are already known. In practice, these estimates can be determined by a calibration process. Some embodiments perform mobility correction during the preprocessing stage. The mobility shifts can be nearly constant across the chosen interval, since they tend to vary on the same scale as the spacing. The mobility shift correction step can be omitted; but it should be appreciated that doing so may increase the likelihood of a poor spacing estimate. Once the shifts are corrected, a single signal can be generated by summing across multiple channels of the electropherogram;

$$y ( t ) = {\sum }_{i} {x}_{i} ( t + δ {t}_{i} ) , \qquad ( 1 )$$

where xᵢ(t) is the i-th channel of the electropherogram at scan t, and δtᵢ is the mobility shift of the i-th channel.

Various embodiments of the system compute a high-pass filtered Fourier transform of the real-valued signal, y(t). High-pass filtering can be performed in either the time or the frequency domain. If a rough estimate of an expected spacing is available, ŝ, filtering in the frequency domain can be advantageous as the filter, Hᵢ(ν), can be easily tuned. The filtered power spectrum is the squared amplitude of the filtered transform result,
p_{ŝ}(ν)=|H_{ŝ}(ν)·Y(ν)|², (2)
where Y(ν) is the Fourier transform of y(t).

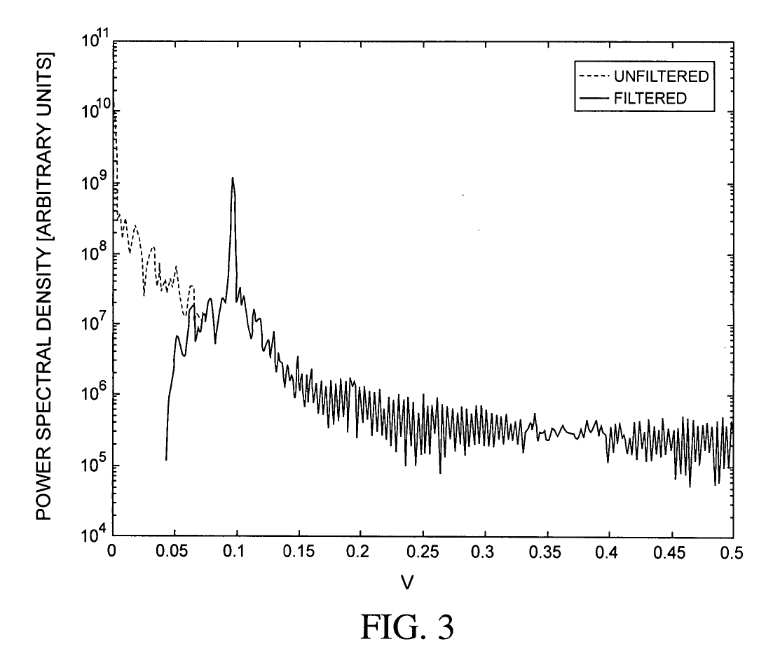

Various embodiments match the high-pass filter approximately to the spacing of the peaks. If the cutoff frequency of the filter is too low, power at low frequencies can be insufficiently attenuated, making it difficult to identify the peak in the filtered power spectrum that represents the average spacing of peaks in the electropherogram. If the cutoff frequency is too high, the spacing peak can be attenuated and rendered indistinguishable from spurious peaks in the higher frequency noise. In general, the cutoff frequency should be about equal to or somewhat smaller than \hat{ν}=ŝ⁻¹. FIG. 3 shows an example of a filtered power spectrum. Here the broken line represents the original spectrum and the solid line represents the filtered spectrum. The filtered spectrum has been tuned to have a cutoff close to the expected spacing.

Location of the global maximum of the high-pass power spectrum can yield the spacing peak. Locating the second highest local maximum can be useful for estimating the reliability of the final result, as sometimes the high-pass power spectrum can possess more than one strong peak.

The dominant peak of the high-pass power spectrum generally characterizes the average peak spacing in the original signal. In such cases, the location of the global maximum, ν₀, is inversely related to the average spacing,
\overline{s}=ν₀⁻¹. (3)
For the example shown in FIG. 3, the maximum occurs at ν₀≈0.096, which implies a spacing of \overline{s}=10.4.

If a rough estimate of the spacing is known, the location of the global maximum can be limited to within an interval corresponding to the expected spacing scale. For example, if one is highly confident that the peak spacing should be between 10 and 25 scans (samples), one would expect the dominant peak in the spectrum to lie in the interval (0.04, 0.10). A global maximum outside that interval can then serve as an indication of the quality of the estimation.

The secondary maximum can be used to measure the reliability of the estimate of average spacing. A secondary maximum that is comparable in amplitude but not frequency can indicate potential for a large error in the estimate. If the secondary maximum is located at ν₁ and has an amplitude of p₁=pᵢ(ν₁); and the amplitude of the global maximum is p₀=pᵢ(ν₀) One can then measure the confidence of the spacing estimate by

$$q = 1 - \frac{{p}_{1}}{{p}_{0}} . \qquad ( 4 )$$

This particular metric is only one example of many that could be used as a heuristic measure of the quality of the estimate. Another example is

$$δ {s}^{2} = {( \frac{δ {ν}_{0}}{{ν}_{0}^{2}} )}^{2} + {( {α [ {ν}_{1} - {ν}_{0} ] [ \frac{{p}_{0}}{{p}_{1}} ]}^{2} )}^{2} , \qquad ( 5 )$$

where δν₀ is the width of the dominant peak, and α is an adjustable parameter used to mix in the heuristic term that depends on the characteristics of the secondary maximum. δs can be taken as the uncertainty of the estimate \overline{s}.

v. Estimation of Peak Width

In base calling systems, it can sometimes be desirable to estimate the typical or average width of peaks in the electropherogram. Various embodiments contemplated herein do not require a priori detection of a signal's peaks. This freedom from peak detection can provide an advantage, for example, when the signal is littered with contamination peaks and instrument artifacts.

According to various embodiments, average width can be estimated through examination of the Fourier transform of the signal. For example, an interval of the data can be selected throughout which the average peak width is approximately constant. For the data in this interval, the following steps can be executed:
1. Compute a spectral function whose form depends on the average peak width
2. Model the spectral function
3. Compute the width corresponding to the model

An individual channel in the electropherogram can be considered to be a composition of delayed Gaussian peaks,

$${x}_{c} ( t ) = {\sum }_{j} {a}_{j , c } g ( t - {t}_{j , c} ) , \qquad ( 6 )$$

where α_{j,c} and t_{j,c} are the amplitude and position of the j-th peak of the channel c, and g(t) is a peak model, with constant width.
The Fourier transform of such a signal is
X_{c}(ν)=f(ν)G(ν), (7)
where G(ν) is the Fourier transform of g(t), and

$$f ( ν ) = {\sum }_{j} {a}_{j , c} {ⅇ}^{2 π {it}_{j , c} ν} .$$

Evaluation of f(ν) can be difficult since both a_{j,c} and t_{j,c} depend on the details of the electropherogram being examined. Analysis can be simplified by the assumption that |f(ν)| tends to vary slowly and results in a large spectral component close to the origin.

Thus X_{c}(ν) often displays strong peaks near the origin and at a location corresponding to the average spacing of the peaks. Analysis can also be simplified by the assumption that base peaks in the electropherogram can be to be Gaussian and can be approximated by the equation

$$g ( t ) \approx exp \frac{- {t}^{2}}{2 {σ}^{2}} \qquad ( 8 )$$

The Fourier transform of g(t) is then also nearly Gaussian
G(ν) αe^{-2πσ}^{²}^{ν}^{²} (9)
where α is a normalization constant. Thus
A_{c}(ν)≡log|X_{c}(ν)|≈−2πσ²ν²+β_{c}, (10)
where β_{c} is a normalization term that depends on α and |f(ν)|.
Equations 6 and 8 are approximations and while these models work well, one skilled in the art may use others. One skilled in the art will appreciate that other models can be used.

Contamination and peaks from the noise space can introduce deviations from the approximation of Equation 9. In particular, noise in the system can cause the computed function A_{c}(ν) to level off for frequencies higher than some \hat{ν}. Furthermore, the spacing feature of f(ν) mentioned previously typically shows up in A_{c}(ν) as a small peak at ν₁=\overline{s}⁻¹, where \overline{s} is the average spacing of peaks in the electropherogram. If the widths of the peaks are close to the same across the channels of the electropherogram, the impact of these anomalies can be reduced to some degree by combining the signals for all channels.

$$A ( ν ) ≡ log {\sum }_{c} \vert {X}_{c} ( ν ) \vert \approx - 2 π {σ}^{2} {ν}^{2} + {β}_{c} , \qquad ( 11 )$$

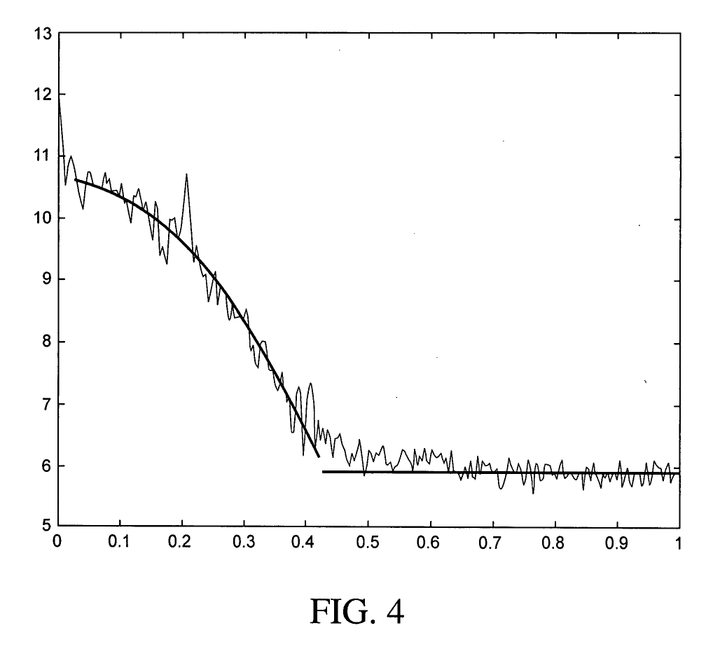

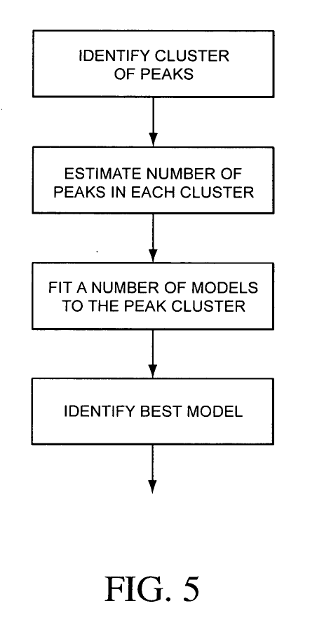

In general, there is a substantial interval of frequencies over which the approximation in Equation 9 is good. A typical example of A(ν) is shown in FIG. 4. Here the thin line represents the spectral function computed from an electropherogram.

In various embodiments, A(ν) can be modeled over the entire range ν. The formulation of the process can be simplified by using the range of the function that is related to the width. As previously mentioned, this can include the region between the slowly varying features and the noise plateau caused any instrument noise. FIG. 4 shows this region as between 0.025 and 0.43. Defining the boundaries of this region as ν₁ and ν₂, this region can be modeled with a quadratic function as follows,
A(ν)_{ν1}^{ν2}≈−2πσ²ν²+β_{c}=B(ν)=−a₂ν²+a₀ (12)
where the term a₂ is equal to 2πσ².

The horizontal line in FIG. 4 represents the noise floor. Many approaches can be used to identify the range ν, to ν₂. In certain embodiments, an iterative approach is employed. The very low-frequency end, of the spectral function, ν<ν₀, can be omitted due to the presence of the ν=0 feature. Then, a frequency, ν₂ which is close to \hat{ν} can be determined. One skilled in the art will appreciate that there are various ways to approximate \hat{ν}. One suitable technique, for example, involves calculating a threshold value
A₂=γA(0)+(1−γ)Â, (13)
where Â is the average of A(ν) over the interval (ζν_{Nyq}, ν_{Nyq}), and where ν_{Nyq} is the Nyquist frequency and γ and ζ are parameters. Suitable values include γ=1/10 and ζ=⅔. Â represents the level of the noise plateau. The approximation ν₂ can be defined as the smallest frequency at which A(ν) crosses the threshold A₂.

The model, B(ν)=−a₂ν²+a₀, can be iteratively fit to the spectral function A(ν) over the range ν₁ to ν₂, until convergence is achieved. The following pseudo code describes an exemplary iterative process:
1. while not converged and ν₀<upper bound for ν₀
   (a) fit model over interval (ν₀, ν₂)
   (b) set ν₃=ν₂ and decrement ν₃
   (c) while not converged and ν₃-ν₀>minimum interval length
      i. fit model over interval (ν₀, ν₃)
      ii. decrement ν₃
   (d) increment ν₀
      The term “convergence” can be used to encapsulate both convergence of the values of the model parameters and fulfillment of any other criteria one might require for an acceptable result. For example, one can require that a goodness of fit metric exceed a threshold value. The exact nature of the increment and decrement operations is also malleable. A new value for ν₀ can be determined by shifting to the next higher frequency estimate in the discretely represented A(ν). A new value for ν₃ can be determined by multiplying by a value slightly less than 1 (e.g., 0.95). The convergence criteria may depend on the exact decrement operation used.
      Estimates of random errors in the calculation of A(ν) can be used in the fitting process. For example, artificially enhancing the errors in a neighborhood of ν, can reduce the impact of the peak-spacing feature.

Once the model has been fitted the width estimate can be calculated using the approximation in Equation 12 yielding,

$$σ = \sqrt{\frac{{a}_{2}}{2 π}} . \qquad ( 14 )$$

vi. Peak Amplitude Model

Many factors in the system can influence how an initial number of fragment molecules of a particular size appear in terms of signal peak amplitude in an electropherogram. For example, the injection process may be preferential to particular fragment sizes or dye labels; the emission response of one dye label may be stronger or weaker than another; etc. One skilled in the art will appreciate that one can generally assume that such effects are possibly dye-specific or slowly varying in the electropherogram and can be normalized via a modeling process.

Various embodiments employ statistical models that model peak amplitudes as a statistical distribution with the estimation of the parameters of the distribution being one step in the overall process. Model estimation can be used to increase the predictive power of a base-calling system. For example, given a section of an electropherogram and a list of detected peaks in that section, a base caller can decide which peaks belong to the target sequence, as opposed to any underlying noise processes that may contaminate the signal. A base calling system that uses prior estimates of signal- and noise-peak amplitude distributions can perform this separation more accurately and can better estimate the probability of error/quality of its resulting base calls.

Some embodiments compute a Peak Amplitude Model (PAM) in a region of the electropherogram where peaks are well resolved. The models can then be used to improve the accuracy of peak detection in regions of the data where peaks are poorly resolved.

PAM estimation can be used to characterize the features of typical DNA sequencing data such as, the local amplitude distribution of peaks in the target DNA sequence (the signal model), one or more distributions of interfering sequences or noise processes (the noise models), constant dye-to-dye differences in signal intensity (the color balance) and slowly varying change in the mean signal and noise intensities over time (the mean signal and noise profiles).

Various embodiments model peak amplitudes observed at a time of arrival t and channel c as a random variable y. In some systems, the PAM can be computed over an interval of the electropherogram that possesses variation in the model parameters with respect to time and channel. Thus, a general model can be described by the probability density function (PDF)f(y;ν), where the vector of model parameters ν may vary as a function of t and c. Some embodiments further formulate the PDF, f, as a mixture model, composed of a signal and one or more noise sources. One skilled in the art will appreciate that this description captures the basic characteristics of the underlying chemistry and enables subsequent calculation of likelihood ratios—i.e., the likelihood that an observed peak originated from the signal, as opposed to a noise, process. The most general case, can be considered to be

$$f ( y ; ν ) = {\sum }_{j = 0}^{M} {w}_{j} ( t , c ) {f}_{j} ( y ; {p}_{j} ( t , c ) ) \qquad ( 2 )$$

where fₒ denotes the PDF of the signal peak distribution, the f_{i≠0} are PDFs that characterize M−1 noise sources, and the weights wⱼ satisfy the relation

$${\sum }_{j} {w}_{j} = 1.$$

Given a model of this nature, the PAM process can compute an optimal fit of the model (i.e., the set of parameters ν=(wⱼ, pⱼ), for j=0, . . . , M) to a set of observed peak amplitudes in an interval of an electropherogram, defined by [τ₁, τ₂].

One skilled in the art will appreciate that after some preprocessing such as normalization to correct for color-balance, slowly-varying mean amplitude time component peaks produced by dye terminator sequencing reactions typically exhibit amplitudes that are well approximated by a lognormal distribution. Some embodiments employ models whose PDF is of the form general form expressed for f(y;ν) but with the substitution y→x=ln(y) and the use of a signal peak model where x is normally distributed, N(η;σ).

The following approximations can be used to constrain the functional form of the signal model parameters p₀(t,c)=(η(t,c), σ(t,c)). The peak amplitudes, y, generally have a global multiplicative scale associated with a channel. For example, a particular dye may have stronger or weaker fluorescent emission. In the log amplitudes x, a multiplicative scale can translate to a constant offset in the mean amplitude η for that channel. Similarly, a slowly-varying mean amplitude is typically observed in sequencing data. This electropherogram profile can be influenced, for example, by the ratio of ddNTPs to dNTPs in the sequencing reaction mix, The assumption can be made that in the log amplitudes x, the time variation is the same across channels, once the color-balance is normalized.

Over a typical short period in the electropherogram, the profile can be approximated over an interval. Typical values for the interval range from approximately 40 to 80 bases. Over the interval, the normal distribution N(η₀; σ₀) can be used to model the log amplitudes x, parameterized in a region [τ₁, τ₂] as η₀(t,c)=a_{0c}, b₀t and σ₀(t,c)=σ_{0c}. The parameter σ_{0c} is generally independent of t, as the raw amplitudes typically have approximately a constant coefficient of variance. The degree to which a varies with c can depend on the details of the sequencing chemistry.

Signal noise can result from electrical sources, such as noise created in the physical light emission and detection processes. This noise scale typically defines the limit of detection and can be generally approximated by a normal distribution. A typical peak detection algorithm locates inflection points and thus even a smoothed background signal with additive electrical noise can produce random peak detections. Examination of the distribution of peaks detected in simulated signals consisting of Gaussian noise run through a smoothing filter has shown that the amplitudes can be approximated by a Weibull distribution. Situations where electrical noise is the dominant noise source are typically rare. Generally, there exist low to moderate levels of peak-like artifacts that arise from one or more chemical processes. This is often referred to as chemistry noise.

One skilled in the art will appreciate that it is a fairly common occurrence for a sequencing reaction to be contaminated with an amount of one or more secondary template molecules. These templates can amplify in proportion during the sequencing reaction and may produce an additional source of secondary sequence noise, observed under the primary peaks in the data.

Some embodiments choose a noise model that includes the latter two noise sources. This approximation can be made by following the assumption that low-level peaks arising from electrical noise can be incorporated at the low end of the chemistry noise distribution. Thus noise can be modeled via a dual-source model, where the log amplitudes are normal distributions and the system can be described by the following conditions, pⱼ(t,c)=(ηⱼ(t,c), σⱼ(t,c)), ηⱼ(t,c)=a_{ic}+bⱼt, σⱼ(t,c)=σ_{jc}, where i={1, 2}1.

The task of fitting a PDF of the general form to a set of observed peak amplitudes can be envisioned as a problem in non-linear optimization. For example, using the preceding formulation the problem becomes one of determining the maximum likelihood estimates of the model parameters (a_{jc}, bⱼ, σ_{jc}) and the weights wⱼ. In the case where j={0, 1, 2}, this leads to a total of thirty parameters. The size of the parameter space can be reduced by imposing additional constraints. Exemplary constraints are the assumption that chemistry noise and secondary sequence noise scale uniformly with the primary sequence peaks and that such scaling assumptions are time independent. These assumptions permit the simplification that the mean log amplitude parameters a_{0c} are equal to the difference in the corresponding parameters a_{jc} for the two noise distributions, j≠0 and bⱼ≡b. Another exemplary constraint involves assuming that σ_{jc}=σⱼ independent of the channel. These assumptions can reduce the parameter set to p=(aⱼ, b, σⱼ, d₂₁, d₃₁, d₄₁), and w=wⱼ, where j=0, 1, 2; and dₖₗ≡a₀ₖ−a₀ₗ thus leaving only 13 parameters. These parameters translate to mean amplitudes aⱼ, the amplitude standard deviations σⱼ, and the mean amplitude slope in the region, b. One skilled in the art will appreciate that other simplifying assumptions can be made in place of or in addition to the aforementioned without departing from the spirit of these teachings.

Some embodiments fit a PAM to smaller, overlapping regions of the electropherogram by dividing the signal into sections. Exemplary section lengths are approximately 40 to 80 bases. The model change point method can be used to perform such sectioning. This strategy can be employed as typical electropherogram signals can exhibit a slowly-varying mean amplitude profile that can be influenced by differences in sequencing protocol (reaction mix, clean-up procedures, etc.) from one experiment to another and it can be difficult to find an analytical model and set of parameters that can be used to characterize the entire profile of an electropherogram.

A fit in one region can be constrained by the results of a fit in a neighboring region. For example, it can be a requirement to have mean amplitude profiles agree at a point in the overlap region. Representative signal and noise distributions can be computed at each point as a weighted average of the overlapping, parameterized models. One skilled in the art will appreciate that this is only one of several possible strategies for fitting the data by sections and that others may be employed without departing from the sprit of these teachings. The PAM fitting process can be summarized by one or more of the following steps:
Divide the data into sections.
For each section,
   Detect peaks; compute their log amplitudes.
   Make initial estimates of the model parameters.
   Compute a fit of model parameters to detected peak amplitudes.
Compute peak amplitude models from the contributing section models.

While a variety of peak detection methods can be employed, one approach that can be effective is to apply a smoothing filter at an appropriate scale and find local maxima in the smoothed signal.

While many different methods of optimizing the model fit can be employed, many non-linear and linear optimization techniques require initial estimates of the parameters. Some embodiments arrive at initial estimates of the model parameters from the electropherogram signals in the interval, independent of any peak detection scheme. This can have the effect of reordering the steps in the PAM fitting process. An embodiment example of such a system can use a morphological filter on a sorted signal. Such a method proceeds by sorting the electropherogram matrix E_{ic} creating a new electropherogram matrix Êₜₖ, so that channel k=0 contains the largest amplitude at each time point, and k=3 contains the smallest. For channels k≈0, cusps, which arise when DNA signal peaks of different channels overlap partially in time, are located and removed from Êₜₖ to obtain Êₜₖ*. For each channel k, the morphological closing filter, Mₕ, is then applied to raise isolated negative features narrower than a given scale h to the level of their neighborhood. Application of the decimation function D, then selects every l^{th} value from the signal, to compute the signals xₖᵢ≡xₖ(tᵢ)=DₗMₕÊₜₖ*. The parameters (a₀, b, σ₀) can be obtained from x₀ᵢ by computing a fit of the n data points x₀ᵢ vs. tᵢ. (a₀, b) can be computed as the intercept and slope respectively of a linear least-squares fit of x₀ᵢ vs. tᵢ. σ₀ can be computed as the standard deviation of the n values given by x₀ᵢ-a₀-btᵢ. In a similar fashion the secondary sequence noise model parameters (a₁, σ₁) can be computed from the values x₁ᵢ, vs. tᵢ. In a similar fashion the chemistry noise model (a₂, σ₂) can be computed using a linear combination of x₂ and x₃. Some embodiments use only x₃ in this computation. The color-balance offsets and distribution weights can all be assigned the value 0 and the weights can all be equal at ⅓.

Some embodiments use peak detection methods in the initial estimation of PAM parameters. An embodiment using this technique sorts a list of input peaks by amplitude, from largest to smallest or alternatively, sorting sections of the interval (e.g., halves or thirds) by amplitude. Based on an estimate of the average spacing \overline{s}, the first n peaks in a section, where n□(τ₂−τ₁)\sqrt{s}, for a section bounded by [τ₁, τ₂] can be selected and an assumption made that the peaks in this subset arise from the signal distribution. The parameters (a₀, b, σ₀) can be fit using a linear fit to these data and these peaks can be removed from the peak list. The process can be repeated for j=1, 2 to estimate the parameters (aⱼ, σⱼ).

One skilled in the art will appreciate that a plurality of optimization techniques such as steepest descent, conjugate gradient or Newton-Raphson methods, can be employed to optimize the model parameters. The selection of the optimization technique can depend on the exact format of the model chosen. Some embodiments use the Expectation-Maximization algorithm and exploit its appropriateness for optimizing mixture models such as the one formulated previously.

Formally stated, the problem can involve maximizing the log likelihood function for the general model,

$$log L ( v ) = {\sum }_{i = 1}^{N} log f ( {o}_{i} ; v ) = {\sum }_{i = 1}^{N} log \{ {\sum }_{j = 0}^{M - 1} {w}_{j} {f}_{j} ( {o}_{i} , {p}_{j} ) \}$$

under the parameters ν=(wⱼ, pⱼ). As previously stated the parameter set can be constrained by various simplifying assumptions. One skilled in the art will appreciate that the details of applying the EM algorithm to such a problem are commonly available through a variety of references such as McLachlan and Krishnan, The EM Algorithm and Extensions, John Wiley & Sons, Inc., 1997 or Cherkassky and Mulier, Learning From Data, John Wiley & Sons, Inc., 1998.

Some embodiments employ an unconstrained model in the maximization phase where 4M separate distributions f_{jc} can be characterized by independent parameters, e.g., p_{jc}=(a_{jc}, b_{jc}, σ_{jc}) and the optimization problem can be solved as 4M independent least-squares problems. Some embodiments constrain the problem during the maximization phase by using only the most recent fit to the signal model f₀ to compute the color balance coefficients d_{0c}. The remaining noise models can be constrained to this color balance, because each iteration always has the most recent color balance removed from the data that contributes to each maximization step.

Some embodiments employ variations on the model and methods of solution. For example, prior estimates of the color balance logs d_{0c} can be made using alternate techniques, such as examining the total power in each channel over a range of the electropherogram. In such a case, the d_{0c} would no longer be parameters of the PAM. Another example of a variation is to make a first pass of the full modeling process on the electropherogram, and compute color balance logs \overline{d}_{0c} as a weighted average of the d_{0c} estimated in each interval of the data. Another example of a variation is to make a second pass of the full modeling process, with the color balance fixed at \overline{d}_{0c} and removed from the log amplitudes. Another modification is to modify the constraining conditions to accommodate more variable signal or noise models such as allowing the noise model slope to be an independent parameter bⱼ or allowing the signal model to accommodate dye-specific variability through independent parameters σ_{0c} or allowing the interval signal or noise models to be higher-degree polynomials or some other function of t. Model variations of this nature can be solved with suitable modifications to the EM iteration steps.

Various embodiments use techniques that enable the optimal estimation of sequence context-dependent amplitude models, or CDAMs, in an a priori training process. In such cases, models characterizing different product sequencing kits can be formed. The CDAM model can be represented as a set of coefficients for chemistry calibration and be input into automated base calling or quality value prediction software to increase accuracy and predictive power.

One skilled in the art will appreciate that the model choice described herein is only one of many possible models. The model and method of determining model parameters can be changed without deviating from the spirit of these teachings.

D. Regularizer

Regularization involves applying the information gained by the calibrator to the signal in order to normalize time dependent effects. This can produce a signal that has relatively uniform amplitude and spacing.

Some embodiments of the system do not include an explicit regularizer. Such systems can still incorporate the paramaterizations afforded by the calibrator by using the information as input to the other sub-processes. One example is the peak detector discussed in section E which uses spacing information. In such cases, the regularizer can be considered to be implicit to the base calling system.

Some embodiments use the regularizer and calibrator iteratively to fine tune parameters. An example of such behavior is an embodiment which performs regularization based on various parameters determined by the calibrator and then uses a matched filter for peak detection. Subsequent application of the PAM process can be used determine the channel color balance parameters. The channels are color balanced and deconvolution is then performed via a technique such as that contemplated in U.S. patent application Ser. No. 10/134,196, assigned to the assignee hereof, which is incorporated by reference herein in its entirety. Widths can be readjusted and the Peak Amplitude Modeling process can be run again to obtain models for the regularized signal.

i. Matched Filter Peak Detector

Many peak detection methods analyze the derivatives of a signal in order to identify peaks. Some implementations can be of such high sensitivity that they result in the identification of numerous peaks. While, such non-signal peaks can be of interest for characterizing the performance of the system, generally these peaks are considered extraneous and may be of little interest or utility in characterizing the underlying signal of interest. Tuning of such algorithms to achieve a suitable balance between the identification of signal and noise peaks can be a difficult process.

The present teachings employ the use of a matched filter prior to analysis by peak detection schemes in order to preselect peaks which can be of more interest to downstream peak classification methods. In some embodiments, a priori knowledge about the shape of expected peaks and can be exploited to design a matched filter which selects for these features in the signal. A correlation between the matched filter and the signal can result in a matched filter signal that contains information communicating the location of prototypical peak-like features in the signal.

The matched-filter process can be used for processing base calling signals by employing one or more of the following steps on the of a signal.
Initialize the matched-filter signal r(j) to 0
Segment the data into one or more segments
For each segment, construct a matched filter that detects peaks corresponding to predefined feature scales. These scale can be determined by peak-model estimation routines or set based on prior knowledge or expectations.
If the signal exceeds a predefined threshold, calculate the value of the normalized matched-filter correlation r(j), otherwise leave r(j)=0.

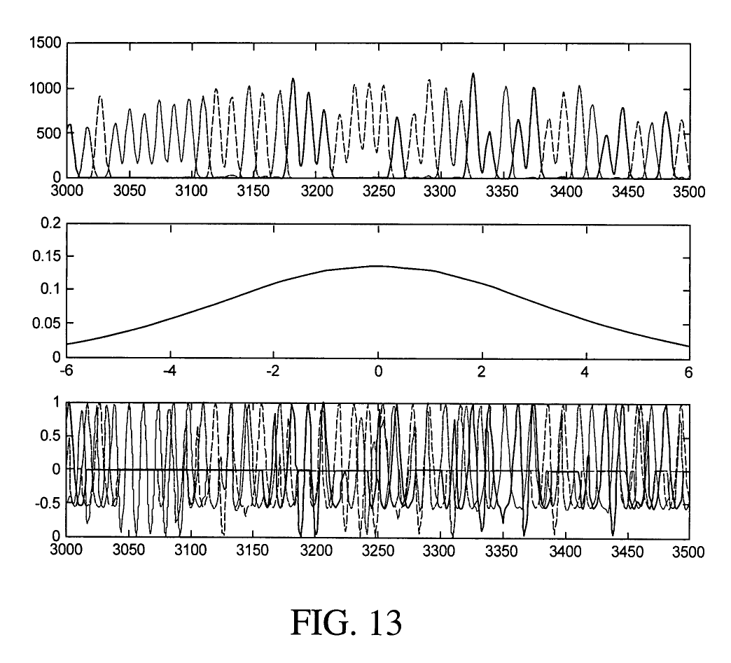

FIG. 13 illustrates the operation of a matched filter on sequencing data. The top panel shows the analyzed signal after some degree of preprocessing. The middle panel represents a truncated Gaussian filter used in some embodiments of a matched-filter system. The Gaussian is described by, f(i)=exp(−k²/2σ²) where i=−k . . . 0 . . . k, k is the half-window size governing the size of the matched filter and σ is the peak width. While a Gaussian is used as the peak model, other peak models may be used while maintaining the spirit of these teachings. The bottom panel shows a normalized correlation signal between the analyzed signal and the Gaussian model. The peaks in the matched-filter signal that are not sample related, arise from low amplitude peaks in the top panel which fit the peak model. Low-amplitude and high-amplitude peak-like features are typically emphasized equally in matched-filter processing, with the result that the matched-filter signal can appear noisier than the original signal. This situation can be overcome by employing an appropriate threshold on the matched-filter signal. This can facilitate tuning for a desired level of sensitivity and the retention of features which are most appropriate for downstream classification. A suitable value for screening low level peaks from sources such as shot noise while retaining peaks from many forms of chemistry noise is 6. The resulting matched-filter signal in the bottom panel contains a series of sharp peaks that indicate where peak features are likely to be located.

Various embodiments employ standard peak detection techniques on the match-filter signal such as the Savitzky-Golay (S-G) peak detector. This algorithm finds peaks by smoothing the data with a S-G filter¹ of a prescribed order and detecting peaks via the location of local maxima. While the tuning of S-G peak detection algorithm used in isolation can be problematic. Some embodiments increase the sensitivity of overall peak detection by coupling peak detection with matched filtering. Tuning can then be effected by adjusting the size of the matched filter. For example, the matched filter shown in FIG. 13 extends for k=12 scan points on either side. This shape matches isolated peaks well. A looser filter, which only extends to several scan points on either side of the maximum, can be used to identify less resolved peaks. Peak detection can be further tuned by changing the thresholding parameter specified to the S-G algorithm. This parameter specifies a minimum amplitude below which the S-G algorithm will not call peaks. For example, a value of rₘᵢₙ=0.4 would be a useful threshold for keeping the best peaks in the matched-filter signal in FIG. 13. In some embodiments where it may be desirable to call more peaks than not, the various sensitivity parameters can be tuned to allow looser peak detection. This approach can enable the peak classifier to better decide which peaks are signal and which are noise. For these purposes, a choice of k=4 and rₘᵢₙ=0.2 can be used.
¹ WH Press, SA Teukolsky, WT Vetterling, BP Flannery. Numerical Recipes in C: The Art of Scientific Computing 2^{nd} edition. Cambridge University Press: Cambridge, England. 1992.

To the extent that the scheme described here can be applied to any one-dimensional feature-detection algorithm, other uses for this method can be envisioned. For example, this method is not limited to only detecting peak-like features; other types of one-dimensional features such as double peaks, peaks with shoulders, or dips can be detected. Matched-filter processing can be used to select for such features.

E. Model Based Peak Detection

One skilled in the art will appreciate that many peak detection methods rely on the analysis of zeros of derivatives of the signal. Such methods can fail when the resolution of the peaks in the signal falls below some threshold, when the estimated position of the detected peak can be shifted by the influence of a nearby overlapping peak or when data is particularly challenging. This latter case often occurs towards the end of a read where the signal can degrade to the point where peaks smear together and lose their distinct nature. Various embodiments contemplated herein employ the Gaussian equation to model the peaks and employ a peak spacing estimate ŝ(t) and a noise estimate. The noise estimate can be determined by a variety of methods and leads to the establishment of a threshold signal level \overline{x}.

In some embodiments, the peak model g(t;α) is centered at t=0 and depends on a set of parameters α=(α₁, . . . , αₙ_{ₐ}). Generally, these parameters also depend on time, and can vary over the duration of the signal. The formulation can be simplified by omitting position and amplitude parameters from the peak model. For example, the Gaussian peak model

$${g}_{Gaussian} ( t ; σ ) = exp \frac{- {t}^{2}}{2 {σ}^{2}} , \qquad ( 16 )$$

possesses only one parameter which relates to peak width.

The model-based peak detection process can be described by the following steps:
1. Identify peak clusters
2. Estimate the number of peaks in each cluster
3. Fit hypotheses to the peak cluster
4. Choose hypothesis to be considered in the classification stage

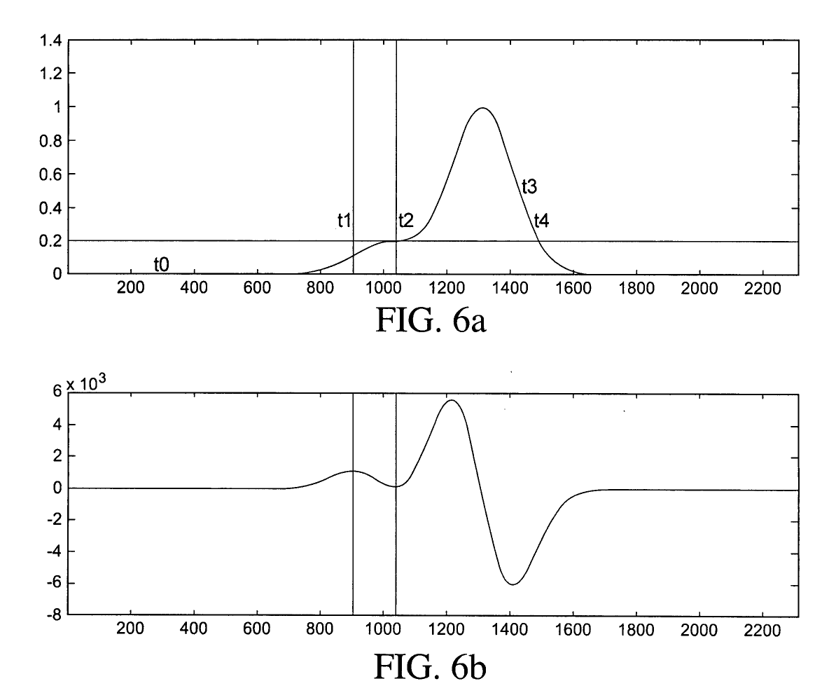

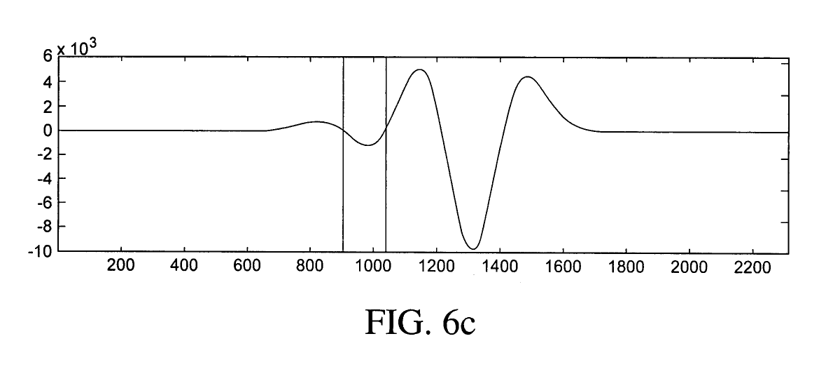

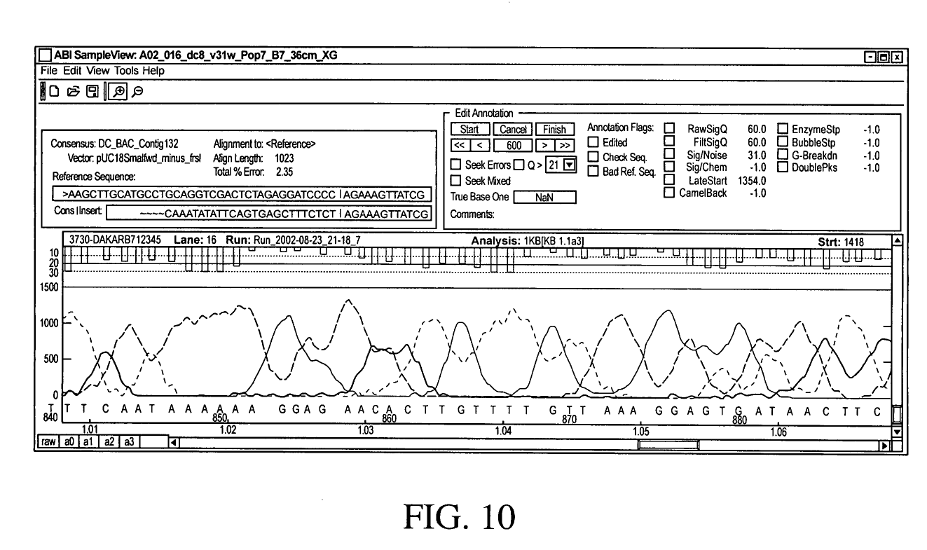

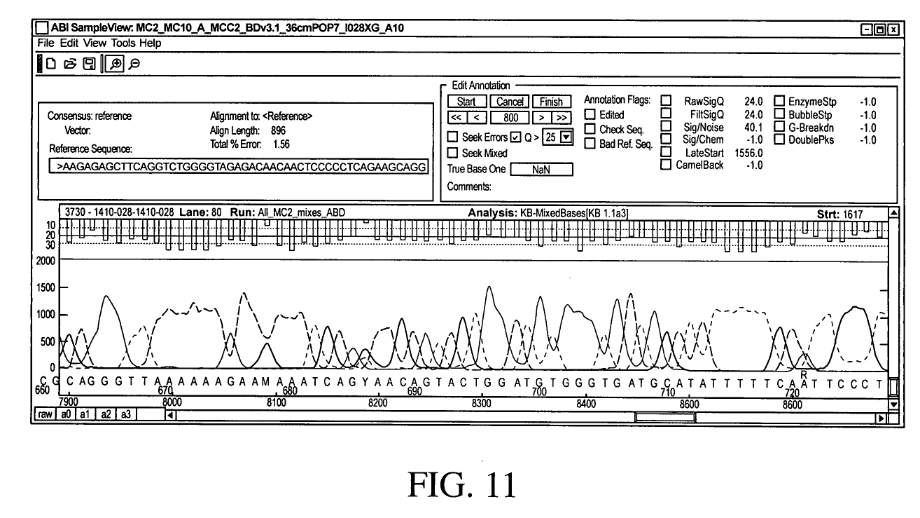

Peak clusters can be comprised of one or more peaks. In general a peak cluster may be thought of as a region in a channel that rises significantly from the noise floor and is distinct from other peak clusters. An example of this can be seen in FIG. 10. The traces in this figure come from a region of data that is poorly resolved. A peak cluster can be seen in the red (T) channel in the region between approximately 10300 scans and 10500 scans. Various embodiments described herein identify a peak cluster by first defining an arbitrary starting point t₀. This point could simply be the beginning of the signal. The method involves scanning forward in time until the signal exceeds the threshold \overline{x}. Then if the second derivative x″(t) of the signal is not positive at this point, scanning proceeds backwards in time until it is positive. In pseudo code, set t₂=t₀
1. while x(t₂)<\overline{x}, increment t₂
2. set t₁=t₂−1
3. while x″(t₁)<0, decrement t₁
   The result, t₁, can be set as the lower bound of the upcoming cluster interval. In a complementary fashion, the end of the peak cluster can be identified. Starting with the value of t₂ resulting from the above search,
1. set t₃=t₂
2. while x(t₃)≧\overline{x}, increment t₃
3. set t₄=t₃
4. while x″(t₄)<0, increment t₄
   Upon completing these two searches, t₁<t₄, and the peak cluster is defined as beginning and ending at these two points. If the end of the signal, or some other arbitrary stopping point, has not been reached, t₀ is set to t₄ and the process can be repeated in order to identify the next cluster interval. FIG. 6 illustrates this process. In FIG. 6a a single peak cluster is present. FIG. 6b shows the first derivative of this signal and illustrates that if a simple zero crossing is used to determine the peak, only the dominant peak would be found. FIG. 6c shows the second derivative and how using it to step backwards will capture the smaller peak that is also present in the peak cluster. The appropriate values to t₀, t₁, t₂, t₃, and t₄ are labeled on FIG. 6a.

Various embodiments estimate the number of peaks in the peak cluster by using a peak model g(t;\hat{α}(\overline{t}) and a spacing estimate ŝ(\overline{t}) where \overline{t} is the location of the peak cluster interval in the electropherogram. \hat{α}(t) and ŝ(t) generally vary slowly and can be considered to be constant over the peak cluster interval. The particular choice of \overline{t} is not critical but choosing it so that t₁≦\overline{t}≦t₄ can result in good results. Typically, one of the elements of a is a peak width parameter. If the peak model is a Gaussian, this can be denoted by the symbol σ, and the assumption that it is constant simplifies the building of hypotheses for the peak cluster composition. Letting σ be a measure of the “half-width” of the peaks, the number of peaks can be estimated from the width estimate \hat{σ}, the spacing estimate ŝ, and the length of the cluster T as follows,

$$\hat{N} = 1 + \frac{T - 2 \hat{σ}}{\hat{s}} . \qquad ( 17 )$$

The length of the cluster is T=t₄−t₁. When using a Gaussian model, the estimate can be improved by exploiting the fact that σ is the distance from the peak center to the inflection points (when the peak is isolated). Letting t₂ and t₃ be the locations of the first and last inflection points in the cluster interval and using T=t₃−t₂ can lead to a good estimate of the number of peaks.

Generally, \hat{N} will not be an integer, and there can be uncertainty or random error in the estimates of spacing and width. An interval of numbers of peaks which is likely to contain the true number of peaks can be generated. If estimates of uncertainty of the spacing and width estimates are available, they can be used to determine a corresponding uncertainty of the estimate of the number of peaks. Various embodiments let δ\hat{N} be the relative uncertainty of N. A range of integer peak counts [N₀, N₁] can be determined by rounding \hat{N}(1+δ\hat{N})^{±1.}

After identification of the peak cluster and the cluster interval (t₁, t₄) that it falls in, and a peak number range [N₀,N₁] for the quantity of peaks that can be found in that interval, models can be fit over the cluster interval and peak number range to more accurately estimate the number of peaks and their locations. If N₁=1, and there is reasonable confidence that the cluster contains only one peak, the peak location can be determined by locating it at the zero crossing in the first derivative. Alternately, or if N₁>1, a composite model with N peaks can be fit to the signal in the cluster interval for each integer value of N in the interval [N₀, N₁].

Various embodiments use nonlinear models for the fitting process. And allow all the peak model parameters αᵢ, positions uᵢ, and amplitudes aᵢ to vary independently for each of the N peaks in the composite model

$${y}_{N} ( t ) = {\sum }_{i = 1}^{N} {a}_{i} g ( t - {u}_{i} ; {α}_{i} ) \qquad ( 18 )$$

This generally implies that the composite model will be a nonlinear function of the model parameters, αᵢ, uᵢ, and aᵢ Constraints can be used on some of the parameters of the composite model in order to simplify it. For example, the amplitudes can be constrained to be non-negative. Additionally, one can add terms that represent available parameter estimates to the objective function of the fit. For example, one could add the term

$${\sum }_{i} {( \frac{{σ}_{i} - \hat{σ}}{δ \hat{σ}} )}^{2} , \qquad ( 19 )$$

to χ² to create a customized objective function to be minimized. Here σᵢ is the width of the i^{th} peak in the composite model, and δ\hat{σ} is the uncertainty of the width estimate \hat{σ}. A constrained nonlinear optimization algorithm can be used to solve such an optimization problem.

Various embodiments reduce the number of parameters by adding constraints to the model. For example, the assumption can be made that the parameters α will vary slowly and can be treated as constant over the cluster interval. Thus the corresponding parameters for different peaks in the composite model can be constrained to be equal αᵢ=α. For example, the widths of the peaks in electropherograms tend to be fairly consistent. So, the widths of the peaks in the composite model can be constrained to be equal; σᵢ=σ.

A similar constraint on the positions of the peaks can be added; uᵢ=u₀+is, where u₀ and s are the remaining independent parameters. In some systems, the spacing of peaks in the electropherograms fluctuate somewhat more than peak widths. In such systems, constraining the positions of peaks in the composite model can be viewed as being aggressive. However, such constraints reduce the complexity of the optimization problem substantially.

Various embodiments use linear composite models in the fitting process. For example the highly constrained composite model,

$${y}_{N} ( t ) = {\sum }_{i = 1}^{N} {a}_{i} g ( t - {u}_{i} ; \hat{α} ( \overline{t} ) ) , \qquad ( 21 )$$

has been used where the u₁ are fixed positions of the peaks composing the model. The only free parameters in this linear composite model are the amplitudes aᵢ. Though it is highly constrained, the linear dependence of this model upon its parameters permits fitting by standard non-iterative algebraic techniques. Singular value decomposition is a robust technique that provides a solution to this linear model-fitting problem.

The peak positions, uᵢ, can be independently estimated in the context of a Gaussian peak model, by using the locations of the first and last inflection points, t₂ and t₃, in the cluster interval. By setting u₁=t₂+\hat{σ} and u_{N}=t₃−\hat{σ}, the remaining peaks can be spaced equally between the first and last position,

$${u}_{i} = {u}_{1} + ( \frac{i - 1}{N - 1} ) ( {u}_{N} - {u}_{1} ) . \qquad ( 22 )$$

This method of selecting the peak positions can lead to a small peak spacing s=(u_{N}−u₁)/(N−1). This can occur when there exists a pair of poorly resolved peaks of which the amplitude of one is much greater than the other. One of the inflection points can then appear close to the position of the smaller peak. To mitigate this problem, s can be compared with ŝ(\overline{t}). If the ratio is smaller than some threshold, u₁ and u_{N} can be adjusted to move the first and last peaks closer to the bounds of the cluster interval before applying Equation 21 to determine the positions of any internal peaks.

For each fitted model y_{N}(t) with fitted parameters β, a fit quality metric q_{N} can be defined. An example of such a metric is,

$${q}_{N} = {D ( {\sum }_{t = {t}_{1}}^{{t}_{4}} {[ x ( t ) - {y}_{N} ( t ) ]}^{2} )}^{- \frac{1}{2}} , \qquad ( 23 )$$

where the unit of time is the sampling period, and D is an estimate of the number of degrees of freedom of the fit. A variety of different forms of quality metric can be used. For example, the value of the objective function used in the optimization process could be used. That function might include terms like Equation 20 for deviations of the fitted model parameters, such as peak width, from the given estimates a or the deviation of peak spacings from the given spacing estimate at s.

The peaks composing the model that exhibits the greatest quality q_{N} can be reported as “detected” peaks. Alternately, some embodiments of the system, can also use the peaks of a top-scoring subset of peak detection hypotheses as alternative hypotheses. Such alternative hypotheses can be considered by subsequent peak classification methods.

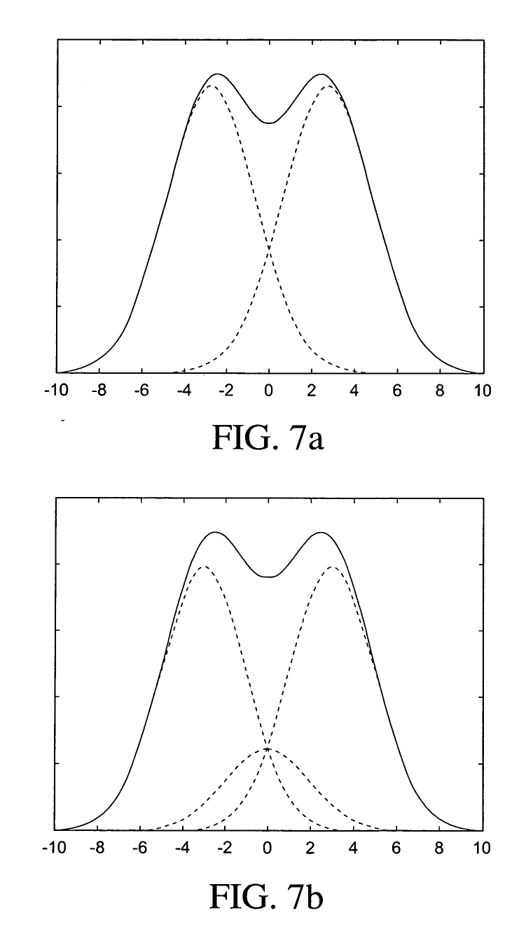

FIG. 7 illustrates how different peak compositions, and hence hypotheses can lead to similar peak clusters. In FIGS. 7a and 7b, the solid line is a peak cluster. These curves are virtually identical. In FIG. 7a, the three peaks represented by broken lines summate to give the peak cluster. The three peaks are Gaussian curves centered at [−3, 0, 3], have amplitudes of [1, 0.31, 1] and have a constant sigma of 2). In FIG. 7b, the peak cluster is the same shape however, it is formed with two Gaussians centered at [−2.73, 2.73], possessing constant amplitude of 1.15 and with constant sigma of 2.12.

F. Peak Classification

Peak classification involves determining if a peak belongs to sample space or if it should be attributed to noise and if the peak belongs to sample space, assigning it a base call. Various embodiments of the system perform this classification, via graph theoretic techniques such as the formation of a directed acyclic graph (DAG). The overall process is sometimes referred to as base calling. The DAG represents possible sequences which can be called using the given peaks.

In various embodiments, each node in the graph corresponds to a detected peak or, if mixed-base identification is desired, a combination of several nearby or coincident peaks. Each detected peak has attributes used by the classification method which include, peak position, peak amplitude, peak color, and peak width. Each edge in the graph, connecting nodes n₁ and n₂, corresponds to the notion that n₂ follows n₁ in the sequence to be base called. Each edge is assigned a weight that corresponds to the relative cost of including this transition in the called sequence.

While a fully connected DAG can be used, various embodiments decrease the complexity of the problem by defining edges only for nodes which are relatively close in scan position. This heuristic significantly can reduce the combinatorial space of possible sequences which could be called from the detected peaks. The traditional shortest-path DAG algorithm is well known in the literature². Given a DAG with non-negative weights, the algorithm determines the shortest (lowest cost) path through the DAG in O(V+E) time using a dynamic programming approach.

Various embodiments employ a modification by proceeding through the sequence of peaks in windowed segments so as to operate on a portion of the peaks at one time. With this adjustment, it is not necessary to specify the topology of a DAG for the entire sequence of peaks before applying the method. Some embodiments consider a window of approximately 40, 50, 60, 70, or 80 times the expected distance between bases. However, one skilled in the art will appreciate that windows of different sizes can be used.

The method is supplied with a sequence of peaks nodes and the identity of the starting node. Some embodiments apply this method on subsequent windows using the last certain node as the starting node for the new window. Some embodiments employ the constraint on the shortest-path DAG algorithm that the last node in the window is automatically included in the shortest path. However, in some cases, this last node may not be a peak that belongs to the sample space and thus should not be included in the final sequence. If the entire global DAG were considered at once, it is more likely that this issue would not arise.

Various embodiments identify the best last node in the current window. One method of doing this employs the following process:
Assign each node an attribute called visitCount. Set this attribute to 0 for each node in the window.
Start at the last node in the window.
Consider the best path to this node:
   For each node in this best path, increment visitCount.
Repeat the preceding step for every node which is close (within a specified threshold) in scan position to the last node in the window.
Identify the node that is closest to the last node in the window that has the maximum value for visitCount. This is the last node to be used in the best path for this window.
   ²T H Cormen, C E Leiserson, R L Rivest. Introduction to Algorithms. The MIT Press, 1990.
   The method works by identifying which node near the end of the window is most used as an ingredient of the best paths. Various embodiments employ the use of phantom nodes and the requirement that these phantom nodes are a part of the final result. For example, one or more phantom nodes can be placed at the end of the window and when the shortest path is found, it will necessarily contain these phantom nodes. However, earlier nodes that result from the signal will be properly considered for inclusion in the shortest path. Once the shortest path is determined, the phantom nodes can be removed.

For each node-to-node transition in the DAG, a weight can be assigned representing the likelihood that the transition should be represented in the sample's sequence. There are many ways to model how the edge weights should be formed. In general any observable or derived parameter can enter the weighting scheme. The observables are not limited to local information, since global information can be accumulated prior to DAG formation. For example, the global spacing and width curves are already known prior to forming the DAG.

In various embodiments the edge weights are modeled as presented herein. For a transition between nodes n₁ and n₂, the edge cost is computed as follows:
Wₜₒₜₐₗ=W_{amplitude}+W_{width}+W_{spacing}+Wₙₒᵢₛₑ (24)
where Wₜₒₜₐₗ is the total cost for the transition; W_{amplitude} can represent the negative log-likelihood that the amplitude of node n₂ represents a signal peak (a base call); W_{width} can be a quadratic cost function which measures the degree to which the width of node n₂ represents a signal peak; W_{spacing} can be a quartic cost function which measures the degree to which the local spacing, including this transition, represents that which would be expected at this position in the trace; and Wₙₒᵢₛₑ can represent a term which penalizes paths which attempt to skip signal-like peaks. These likelihoods can be computed using values which are appropriate for the current location in the electropherogram trace. The semi-local nature of the method can be employed in a variety of ways, such as the incorporation of knowledge about sequence-context amplitude patterns. The Wₙₒᵢₛₑ term can be implemented analogous to the W_{amplitude} term. This term can be defined as roughly equal to the sum of the W_{amplitude} terms for each purported noise peak between nodes n₁ and n₂. The effect of this term is to assign a high cost to paths which attempt to skip signal peaks. In various embodiments the edge weights are allowed to depend on the details of the best path, which leads to the current node. This context information can allow the method to employ semi-local information

The above description of the cost functions can be modified slightly to handle the case of composite nodes. These are nodes which represent putative mixed-base calls. The amplitude cost can include an adjustment which allows for the fact that the primary peak in mixed bases is diminished compared to surrounding peaks, the width cost can be modified to count the most extreme width as the “width” of the node, the spacing cost can use the average spacing between all of the peaks in the each node, and the noise cost can be constrained to consider peaks as noise only those that are not part of composite nodes n₁ or n₂.

The nature of the composite node concept can be constructed such that it is impossible to “double-count” the same peak as both a pure base call and as a member of a mixed base call. This can correct a common defect that is present in other mixed-base calling systems.

The method can also include the ability to consider multiple peak hypotheses presented by the peak detector for peak clusters. It is often difficult to resolve the correct number of peaks in low-resolution clusters of peaks in the same channel. Various embodiments can alleviate this concern by hypothesizing several different numbers of peaks. It is possible for the peak classifier method to use these hypotheses. Use of the peak hypotheses can be effected by not allowing transitions between peaks that are in the same color and in the same unresolved peak cluster, but in different hypotheses.

Various embodiments of the peak classifier can also keep track of sub-optimal paths for traversing the current segment of the DAG. This is an extension of the original optimal path algorithm, with the modification of having each node maintain a list of optimal best-path costs and optimal predecessor nodes instead of maintaining a single value for each. This information can be employed to suggest alternative base calls or to feed information about the method's confidence to a quality-value estimation algorithm.

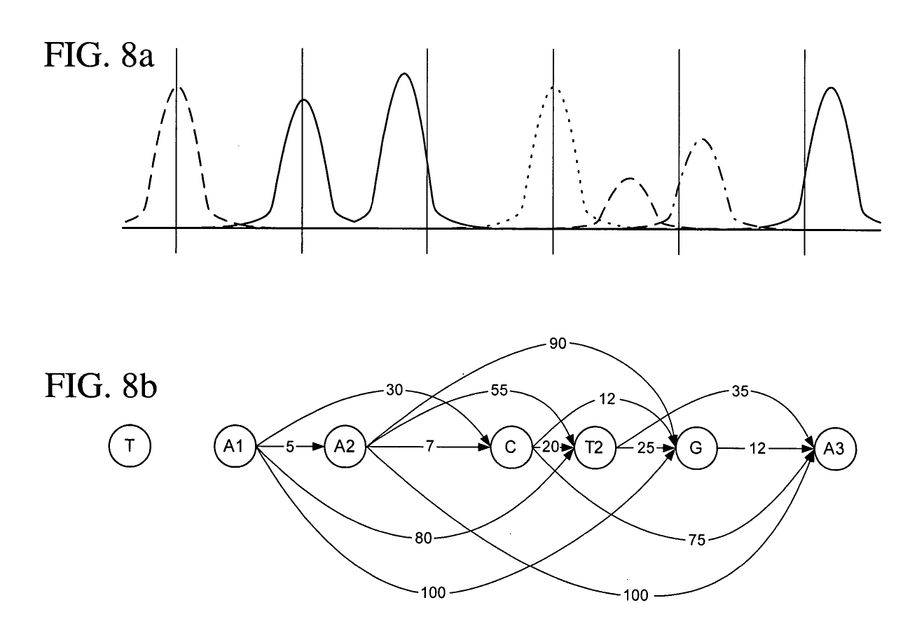

FIG. 8 illustrates an embodiment contemplated the present teachings. It illustrates the formation of a directed acyclic graph and the determination of the shortest path through the graph. FIG. 8a shows the electropherogram data. The x-axis is in units that reflect different mobilities between peaks such as scans, time, bases or other similar scales. The y-axis is in the units of intensity. The vertical lines denote the locations of expected peaks. The peaks are shown in lines of different types. Each type reflects a different channel in the electropherogram. In some embodiments of the system, the cost functions associated with the edges of the graph can be formulated so that a lower score indicates a preferred path. The problem then becomes determining a path that results in the lowest score. The nodes in FIG. 8 represent peaks that are detected during the peak detection stage. It is not necessarily known beforehand if these peaks result from the presence of a nucleotide in the sample or can be attributed to noise (instrument, chemistry, contamination, etc). The figure shows a window of six peaks although one skilled in the art will recognize that a variety of window sizes can be used.

Inside this window in FIG. 8 are six peaks that reflect A, A, C, T, G and A nucleotides. The transition costs are encoded as edge weights. These weights can be formed via a weighted sum of various parameters such as peak amplitude, width, spacing, noise or other peak, sequence context, or semi-local characteristics. For example, the edge weight for the transition between A1 and A2 is fairly low (5) due to the fact that the peak shows good peak characteristics and is located close to an expected position. This can be contrasted with the transition cost for base calling A1 and C with the exclusion of A2. Here the transition cost is high (30) as such a transition would require skipping an expected base entirely. As another example, the edge weight for the C to T2 transition is relatively high (20) as such a transition would require that a peak be base called in a location where a peak is not expected. The path that yields the lowest score visits nodes A1, A2, C, G, A3 and results in a score of 36. Any other path would yield a larger score. For example the path A1, C, T, G, A3 yields a score of 87.

Some embodiments do not fully connect all nodes within the window and retain links only within a moving region within the window. For example, only links representing approximately two to three times the expected spacing between bases may be retained.

For example, if the local spacing is 12 scans/base, then links as far away as approximately 24-36 scans may be retained. The number of nodes considered can depend on the noise level of the sample. Noisier samples have many more spurious peaks and thus more nodes per scan, and thus more links and nodes would be considered in that region. For example, if the data has approximately 5-7 spurious peaks per base, and the local spacing is 12 scans/base then connections between approximately 10-21 peaks would be considered at one time.

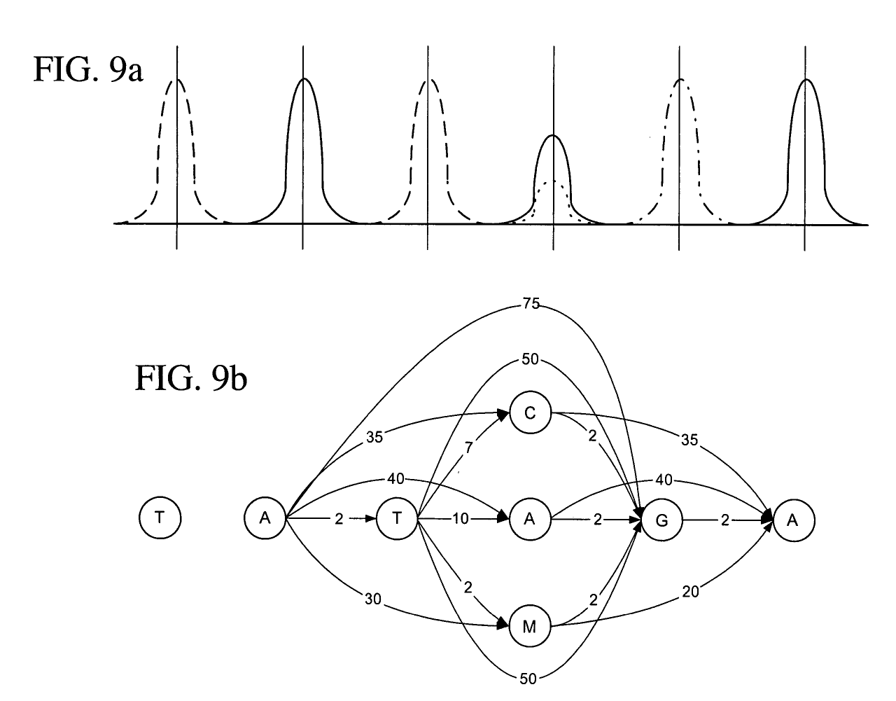

According to various embodiments, FIG. 9 illustrates the creation of a DAG that includes putative mixed-base information. Formation of the DAG follows a process similar to that in FIG. 8. FIG. 9a shows the electropherogram data. FIG. 9b shows the DAG. At expected peak location 5, two peaks exist. The solid trace reflects the channel that corresponds to an adenine nucleotide. The dotted trace reflects the channel that corresponds to a cytosine nucleotide. An additional node is inserted into the DAG to reflect the possibility of a mixed base at this location. This node is designated M. The shortest path through the DAG is ATMGA and has a score of 8.

One skilled in the art will appreciate that for small window sizes such as the size used in the example of figure, an exhaustive search of all possible paths can be executed relatively quickly. When larger window sizes are used, it may be preferable to use a more sophisticated algorithm such as the optimal shortest path algorithm as presented in Introduction to Algorithms (T H Cormen, C E Leiserson, R L Rivest. The MIT Press, 1990). Other algorithms such as the popular Viterbi algorithm as described in Communication Systems (S Haykin. John Wiley and Sons, 2001) may also be used.

A method of checking the accuracy of the sequence produced by the classification is to run the peak list sequence through the classification system in reverse and determine if the same bases are assigned to the sample space. When performing this step, certain local effects that are built into the edge weights may have to be recalculated as they can be direction sensitive.

G. Post Processing

Post processing typically refers to the application of methods of analysis to be completed after the some objective has been completed. Post processing can include several steps such as mixed-base recalling and quality value calculation. Once the bases have been called, some embodiments perform quality value calculation. Quality values can be useful in many secondary analysis methods such as sequence assembly where it is important to ascertain the confidence in a base call in order to determine sequence alignment integrity. The quality values can be determined on a per-base basis or after some number of bases are called.

Various embodiments analyze the called sequence for repetitious patterns that may be indicative of noise or sequence artifact. Insight into correlations between bases can be used for recalling dubious base calls.

Some embodiments determine the confidence, Q, of a called base as a function of several quantities, νᵢ, derived from the electropherogram in the neighborhood of the feature representing the called base. Often the quantities νᵢ are referred to as trace parameters. They may also be collectively referred to as a feature vector v=(νᵢ). Some methods for establishing the function Q(v) involve calibrating or training on a large set of representative annotated data, for which the true sequence is known. One such method was developed by Ewing and Green as referenced in Genome Research, 8 186-194, 1998. In some embodiments, such methods for determining Q(v) are independent of the actual definition of the feature vector v, though they may place constraints on it. An example of such a constraint is that the probability of error can be an increasing function of any individual trace parameter νᵢ.

In some embodiments, parameters determined in the base calling system can be used in the formation of νᵢ. For example, if a graph-theory approach is used as a classifier in the assignment of peaks to noise and sample space, the edge weights can be used to contribute in whole or in part to the feature vector, v. Edges in such a classifier can represent a cost that is related to the likelihood of one node in the graph being a true base call given that the previous node is a true base call and the task of such a base caller is to determine which path from a starting node to a finishing node, (which may or may not be the last node in the graph), has the smallest total cost. Since the feature classification method minimizes the path cost to determine the called sequence, the path cost can be a useful predictor of the probability of an error being made by the classification method.

In some embodiments, the cost of an edge leading to or away from a called base, or some function of thereof, can be used as a trace parameter. Some embodiments incorporate into the trace parameter edge weights that connect prior nodes, or some function thereof. For example, if cⱼ represents the cost of the edge leading to the j-th called base, some embodiments use the following formulation to obtain a trace parameter,

$${u}_{j} = {\sum }_{k} {h}_{k } {c}_{j - k} ,$$

where h=(hₖ) is the impulse response of a FIR low-pass filter. One candidate for the filter is h=( . . . , 0;½,½,0, . . . ), where h₀ is the first element following the semicolon. This filter reflects the premise that an error might be associated with the called base from which an edge leads away. Another possibility for the filter coefficients can be h=( . . . , 0,¼;½,¼, 0, . . . ).

Some embodiments use edge cost functions that depend on relative quantities such as the deviation of the spacing between a particular pair of peaks from the average peak spacing and not on absolute quantities such as the absolute spacing of electropherogram features. This can assist in ensuring that edge cost functions exhibit a consistent scale among samples of equal data quality.

ii. Mixed-base false positive reductions

Some embodiments reduce the number of false positives that can occur during mixed base calling when reproducible sequence noise patterns are present. A false positive in the context of mixed-base base calling is a base which has been assigned a mixed-base base call but which is truly a pure base in the correctly annotated sequence. The combination of reproducible sequence-context amplitude patterns and reproducible sequence noise can be used to identify when mixed-bases are likely to be falsely called and subsequently and can be recalled as pure base calls. A pattern-recaller method can be applied after peak classification or any another base caller technique.

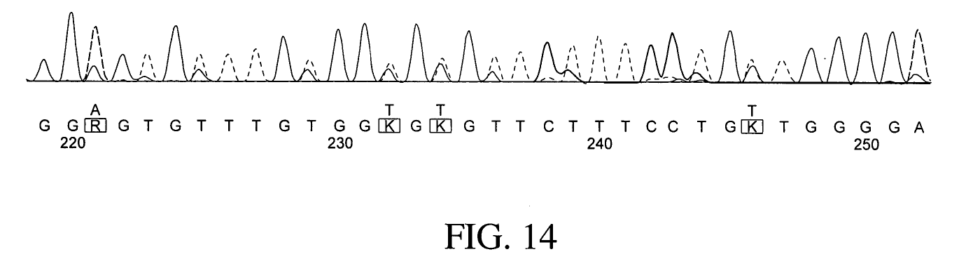

A mixed-base dinucleotide is a dinucleotide sequence which matches the regular expression pattern/[ACGT][RYKMSW]/. The dinucleotide type is the specific base calls of that dinucleotide. For example AM is of a different type than CS. A heavy presence of dinucleotides of this form (for example, a large frequency of AM combinations) can be indicative of combined sequence-noise/sequence-context amplitude problems. This situation is illustrated in FIG. 14 where every G peak is followed by a prominent secondary G peak. Also, because of sequence-context amplitude effects, every T peak that follows a G peak is diminished in amplitude. The combination of these two effects leads to a high number of Ks (T/G) being called in the final sequence. A recalling method can detect the high frequency of GK dinucleotides and recall the Ks which are of poorer quality.

Some embodiments perform recalling. This can be effected by scanning the whole sequence and recording the dinucleotide frequency for each dinucleotide pair and sorting them by the quality of the heterozygote base in the pair and indexed by dinucleotide pair type. If the frequency of a dinucleotide pairs exceeds some predefined threshold, then a pre-defined percentage of the mixed-base calls with the lowest quality values are recalled as pure bases. Typical values for the threshold and percentage to recall are 3 and 66% respectively. Other values can be used depending on the level of aggressiveness desired.

The selected bases for recall can be recalled several different ways. Some embodiments use the color of the primary peak to identify which pure base to call. Various embodiments use peak modeling information and other information which is available from other sources such as peak models, context dependence and global sequence parameters such as noise and spacing.

H. Computer system implementation

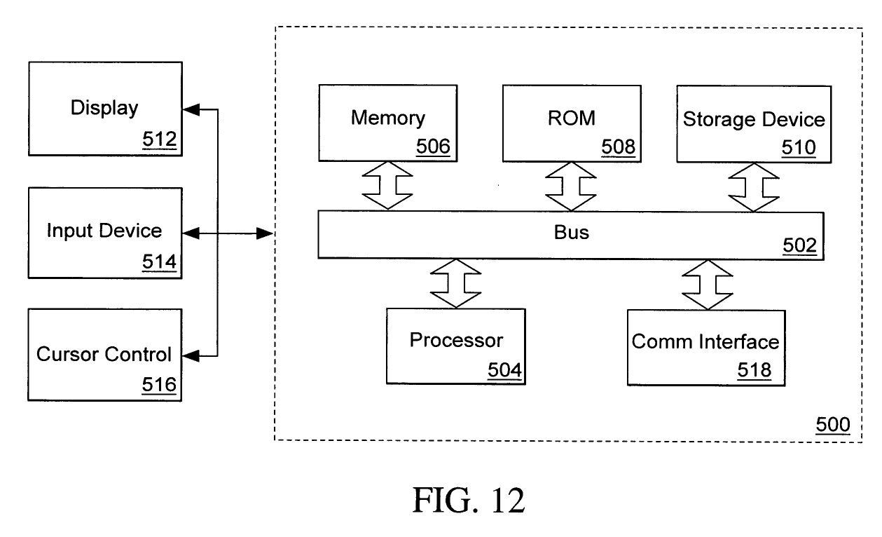

FIG. 12 is a block diagram that illustrates a computer system 500, according to certain embodiments, upon which embodiments of the invention may be implemented. Computer system 500 includes a bus 502 or other communication mechanism for communicating information, and a processor 504 coupled with bus 502 for processing information. Computer system 500 also includes a memory 506, which can be a random access memory (RAM) or other dynamic storage device, coupled to bus 502 for determining base calls, and instructions to be executed by processor 504. Memory 506 also may be used for storing temporary variables or other intermediate information during execution of instructions to be executed by processor 504. Computer system 500 further includes a read only memory (ROM) 508 or other static storage device coupled to bus 502 for storing static information and instructions for processor 504. A storage device 510, such as a magnetic disk or optical disk, is provided and coupled to bus 502 for storing information and instructions.

Computer system 500 may be coupled via bus 502 to a display 512, such as a cathode ray tube (CRT) or liquid crystal display (LCD), for displaying information to a computer user. An input device 514, including alphanumeric and other keys, is coupled to bus 502 for communicating information and command selections to processor 504. Another type of user input device is cursor control 516, such as a mouse, a trackball or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane. A computer system 500 provides base calls and provides a level of confidence for the various calls. Consistent with certain implementations of the invention, base calls and confidence values are provided by computer system 500 in response to processor 504 executing one or more sequences of one or more instructions contained in memory 506. Such instructions may be read into memory 506 from another computer-readable medium, such as storage device 510. Execution of the sequences of instructions contained in memory 506 causes processor 504 to perform the process states described herein. Alternatively hard-wired circuitry may be used in place of or in combination with software instructions to implement the invention. Thus implementations of the invention are not limited to any specific combination of hardware circuitry and software.

The term “computer-readable medium” as used herein refers to any media that participates in providing instructions to processor 504 for execution. Such a medium may take many forms, including but not limited to, non-volatile media, volatile media, and transmission media. Non-volatile media includes, for example, optical or magnetic disks, such as storage device 510. Volatile media includes dynamic memory, such as memory 506. Transmission media includes coaxial cables, copper wire, and fiber optics, including the wires that comprise bus 502. Transmission media can also take the form of acoustic or light waves, such as those generated during radio-wave and infra-red data communications.

Common forms of computer-readable media include, for example, a floppy disk, a flexible disk, hard disk, magnetic tape, or any other magnetic medium, a CD-ROM, any other optical medium, punch cards, papertape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 504 for execution. For example, the instructions may initially be carried on magnetic disk of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector coupled to bus 502 can receive the data carried in the infra-red signal and place the data on bus 502. Bus 502 carries the data to memory 506, from which processor 504 retrieves and executes the instructions. The instructions received by memory 506 may optionally be stored on storage device 510 either before or after execution by processor 504.

The foregoing description of an implementation of the invention has been presented for purposes of illustration and description. It is not exhaustive and does not limit the invention to the precise form disclosed. Modifications and variations are possible in light of the above teachings or may be acquired from practicing of the invention. Additionally, the described implementation includes software but the present invention may be implemented as a combination of hardware and software or in hardware alone. The invention may be implemented with both object-oriented and non-object-oriented programming systems.

The section headings used herein are for organizational purposes only and are not to be construed as limiting the subject matter described in any way.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. A method for determining the sequence of a nucleic acid polymer, comprising the steps of,
obtaining data traces from a plurality of channels of an electrophoresis detection apparatus wherein the traces represent the detection of labeled nucleotides,
preprocessing the data traces,
identifying a plurality of peaks in the data traces,
applying a graph theory formulation to classify one or more of the peaks, and
reporting the peak classifications.

2. The method of claim 1 further comprising,
assigning a quality value to one or more of the classified peaks, and
reporting the quality value.

3. The method of claim 1 wherein the graph theory formulation involves,
using a window to select a portion of the detected peaks,
designating each selected peak as a node in a directed acyclic graph,
connecting the nodes with edges wherein the edges encode a transition cost between the connected nodes, and
classifying the peaks by determining a shortest path through the graph.

4. The method of claim 3 wherein the window encompasses approximately fifty times the expected distance between bases.

5. The method of claim 3 wherein the transition cost is based on at least one of the following characteristics, peak amplitude, peak width, peak spacing, noise, and context information.

6. The method of claim 3, further comprising,
creating an additional node when two or more nodes are within a specified distance so as to appear coincident, and
designating the additional node as a mixed base that encompasses some combination of the coincident peaks.

7. A program storage device readable by a machine, embodying a program of instructions executable by the machine to perform method steps for determining the sequence of a nucleic acid polymer, said method steps comprising:
obtaining data traces from a plurality of channels of an electrophoresis detection apparatus wherein the traces represent the detection of labeled nucleotides,
preprocessing the data traces,
identifying a plurality of peaks in the data traces,
applying a graph theory formulation to classify one or more of the peaks, and
reporting the peak classifications.

8. The device of claim 7 further comprising,
assigning a quality value to one or more of the classified peaks, and
reporting the quality value.

9. The device of claim 7 wherein the graph theory formulation involves,
using a window to select a portion of the detected peaks,
designating each selected peak as a node in a directed acyclic graph,
connecting the nodes with edges wherein the edges encode a transition cost between the connected nodes, and
classifying the peaks by determining the shortest path through the graph.

10. The device of claim 9 wherein the window encompasses approximately fifty times the expected distance between bases.

11. The device of claim 9 wherein the transition cost is based on at least one of the following characteristics, peak amplitude, peak width, peak spacing, noise, and context information.

12. The device of claim 9, further comprising,
creating an additional node when two or more nodes are within a specified distance so as to appear coincident, and
designating the additional node as a mixed base that encompasses some combination of the coincident peaks.
